SEQUENCE LISTING (1 sequence)

SEQ ID NO: 1 - DNA, 12 bp, Artificial Sequence
  synthetic BcgI recognition sequence
cgannnnnntgc

(19) United States
(12) Patent Application Publication
(10) Pub. No.: US 2010/0136559 A1
(43) Pub. Date: Jun. 3, 2010

(54) CHROMATIN STRUCTURE DETECTION

(75) Inventors: Steve Okino, San Carlos, CA (US); Man Cheng, San Ramon, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 12/618,076

(22) Filed: Nov. 13, 2009

Related U.S. Application Data
(60) Provisional application No. 61/119,280, filed on Dec. 2, 2008.

(51) Int. Cl. C12Q 1/68

(52) U.S. Cl. 435/6

(57) ABSTRACT

The present application provides methods and compositions for determining accessibility of a DNA modifying agent in genomic DNA.

### CROSS-REFERENCE TO RELATED PATENT APPLICATIONS

The present application claims benefit of priority to U.S. Provisional Patent Application No. 61/119,280, filed Dec. 2, 2008, which is incorporated by reference for all purposes.

### BACKGROUND OF THE INVENTION

Most DNA in a cell is packaged around a group of histone proteins in a structure known as a nucleosome. This nucleosomal DNA can be further packaged into coiled structures that tightly compact the DNA. This tight packaging can limit the access of DNA to transcription factors and the transcriptional machinery. Genomic DNA packaged in this way is sometimes referred to as chromatin.

Chromatin is classified into two main groups, euchromatin, where the DNA is loosely packaged, accessible and generally, but not always, transcriptionally competent, and heterochromatin, where the DNA is tightly packaged, inaccessible and generally, but not always, transcriptionally silent.

What controls the transition between these two chromatin states is epigenetics. There are two main epigenetic events: DNA methylation and histone modification. These events affect how the DNA is packaged and whether the DNA is active or silent with respect to transcription.

### BRIEF SUMMARY OF THE INVENTION

The present invention provides for methods for analyzing chromosomal DNA, including but not limited to, determining the accessibility of a DNA region on a chromosome to a DNA modifying agent, optionally correlating the accessibility to chromatin structure. In some embodiments, the method comprises:
a. simultaneously:

i. permeabilizing or disrupting a cell membrane of a cell; and

ii. contacting the cell with a DNA cleaving or modifying agent under conditions such that the agent cleaves or modifies the genomic DNA in the cell, wherein different regions of the genomic DNA are cleaved or modified to a different extent by the agent, thereby generating cleaved and intact DNA regions, or modified and unmodified DNA regions; and
b. detecting a physical characteristic or quantity of at least one intact or unmodified or modified DNA region or cloning, isolating, or nucleotide sequencing at least one intact or unmodified or modified DNA region.

In some embodiments, the method further comprises correlating the quantity to chromatin structure of the DNA region in the cell.

In some embodiments, the method comprises detecting a physical characteristic or quantity of at least a first chromosomal intact or unmodified or modified DNA region and the second chromosomal intact or unmodified or modified DNA region; and comparing the physical characteristic or quantity of the first and second DNA regions.

In some embodiments, the method comprises quantifying the number of intact copies of a first chromosomal DNA region and a second chromosomal DNA region, thereby assessing the relative accessibility of the first and second DNA regions to the DNA cleaving agent.

In some embodiments, the genomic DNA is isolated after the contacting step and before the detecting step.

In some embodiments, the cells are permeabilized and contacted with the DNA cleaving or modifying agent while the cells are directly or indirectly adherent to an artificial culture surface.

In some embodiments, the permeabilizing step comprises contacting the cell with an agent that permeabilizes the cell membrane. In some embodiments, the agent that permeabilizes the cell membrane is a lysolipid. In some embodiments, the lysolipid is lysophosphatidylcholine.

In some embodiments, the permeabilizing or disrupting step comprises disrupting the cell membrane with a nonionic detergent. In some embodiments, the nonionic detergent is selected from the group of NP40, Tween20 and Triton X-100.

In some embodiments, the cell is contacted with a DNA cleaving agent, and the DNA cleaving agent is an enzyme.

In some embodiments, the cell is contacted with a DNA modifying agent, and the DNA modifying agent is selected from the group consisting of a methyltransferase and a DNA modifying chemical. In some embodiments, the DNA cleaving agent is a DNA cleaving enzyme; the modifications in the genomic DNA are cleavage sites; and the detecting step comprises quantifying the amount of intact copies of a DNA region after cleavage. In some embodiments, the detecting step comprises quantitative amplification. In some embodiments, quantitative amplification is selected from the group consisting of a quantitative polymerase chain reaction (optionally real-time) and a quantitative ligation mediated polymerase chain reaction (optionally real-time).

In some embodiments, the DNA cleaving agent is selected from a DNase and a restriction enzyme. In some embodiments, the DNA modifying agent is a methyltransferase; the modification is a methyl group on a nucleotide in a nucleotide sequence that would not be methylated by the native methylation enzymes of the cell; and the detecting step comprises a method of detecting the presence or absence of the modification. In some embodiments, the methyltransferase is DAM methyltransferase and the quantifying step comprises cleaving the modified DNA with a restriction enzyme that recognizes a DNA sequence methylated by DAM methyltransferase. In some embodiments, the methyltransferase adds methyl moieties to cytosines in DNA and the detecting step comprises cleaving the modified DNA with a methylation-specific restriction enzyme and/or treating the modified DNA with bisulfite.

In some embodiments, the DNA modifying agent is a molecule having steric hindrance relative to chromatin structure, wherein the molecule is linked to a DNA modifying chemical. In some embodiments, the DNA modifying chemical is selected from the group consisting of dimethyl sulfate and hydrazine.

In some embodiments, the detecting step comprises quantifying at least a portion of a target DNA region and at least a portion of a control DNA region, wherein the control DNA region comprises a sequence that is either
i. accessible in essentially all cells of an animal; or
ii. inaccessible in most cells of an animal; or
iii. with variable accessibility depending on type of cells or growth environment.

In some embodiments, the control DNA region is quantitatively amplified using each of:
primers that prime amplification of a portion of the control DNA region that does not include a potential modification site of the DNA modifying agent; and
primers that prime amplification of a portion of the control DNA region that does include at least one potential modification site of the DNA modifying agent.

In some embodiments, the physical characteristic is DNA methylation.

In some embodiments, step a. comprises contacting the DNA with a DNA cleaving agent, and step b. comprises contacting the intact DNA with bisulfite.

In some embodiments, the method further comprises determining the melting temperature of the bisulfite-treated DNA and correlating the melting temperature to the presence, or absence, or extent of DNA methylation.

In some embodiments, step b comprises preparing a library of: intact DNA regions; or modified DNA regions; or unmodified DNA regions.

In some embodiments, step b comprises contacting at least one intact or unmodified or modified DNA region to a library of polynucleotides under conditions to allow for hybridization of the DNA region to one or more members of the library and detecting hybridization of the DNA region to the one or more members. In some embodiments, the library is organized on a microarray.

In some embodiments, step b comprises amplifying at least one intact or unmodified or modified DNA region.

In some embodiments, step b comprises nucleotide sequencing at least one intact or unmodified or modified DNA region.

The present invention also provides for kits for determining the accessibility of a locus on a chromosome to a DNA modifying agent. In some embodiments, the kit comprises a cell membrane permeabilizing or disrupting agent; and a restriction enzyme, a DNase, or other DNA modifying agent.

In some embodiments, the cell membrane permeabilizing or disrupting agent and the restriction enzyme or DNase are in the same container in the same buffer.

In some embodiments, the cell membrane permeabilizing or disrupting agent and the restriction enzyme or DNase are in separate containers.

In some embodiments, the kit further comprising bisulfite.

In some embodiments, the kit further comprises a primer pair for amplification of a region of genomic DNA of a eukaryote.

In some embodiments, the kit further comprises
a. primers that prime amplification of a portion of a control DNA region that does not include a potential modification site of the DNA modifying agent; and/or
b. primers that prime amplification of a portion of a control DNA region that does comprise at least one potential modification site of the DNA modifying agent.

In some embodiments, the kit comprises
a. primers that prime amplification of a portion of a control DNA region that is not accessible to the modifying agent; and/or
b. primers that prime amplification of a portion of a control DNA region that is accessible to the modifying agent.

Other embodiments of the invention will be clear from a reading of the remainder of this document.

### Definitions

“Permeabilizing,” a cell membrane, as used herein, refers to reducing the integrity of a cell membrane to allow for entry of a modifying agent into the cell. A cell with a permeabilized cell membrane will generally retain the cell membrane such that the cell's structure remains substantially intact. In contrast, “disrupting” a cell membrane, as used herein, refers to reducing the integrity of a cell membrane such that the cell's structure does not remain intact. For example, contacting a cell membrane with a nonionic detergent will remove and/or dissolve a cell membrane, thereby allowing access of a modifying agent to genomic DNA that retains at least some chromosomal structure.

A “DNA modifying agent,” as used herein, refers to a molecule that alters DNA in a detectable manner. Exemplary modifications include DNA nicking or cleavage or introduction or removal of chemical moieties from the DNA. DNA modifying agents that do not result in DNA cleavage include, but are not limited to, DNAmethylases.

A “DNA region,” as used herein, refers to a target sequence of interest within genomic DNA. The DNA region can be of any length that is of interest and that is accessible by the DNA modifying agent being used. In some embodiments, the DNA region can include a single base pair, but can also be a short segment of sequence within genomic DNA (e.g., 2-100, 2-500, 50-500 bp) or a larger segment (e.g., 100-10,000, 100-1000, or 1000-5000 bp. The amount of DNA in a DNA region is sometimes determined by the amount of sequence to be amplified in a PCR reaction. For example, standard PCR reactions generally can amplify between about 35 to 5000 base pairs.

A different “extent” of modifications refers to a different number (actual or relative) of modified copies of one or more DNA regions between samples or between two or more DNA regions in one or more samples. For example, if 100 copies of two DNA regions (designated for convenience as “region A” and “region B”) are each present in chromosomal DNA in a cell, an example of modification to a different extent would be if 10 copies of region A were modified whereas 70 copies of region B were modified.

The terms “oligonucleotide” or “polynucleotide” or “nucleic acid” interchangeably refer to a polymer of monomers that can be corresponded to a ribose nucleic acid (RNA) or deoxyribose nucleic acid (DNA) polymer, or analog thereof. This includes polymers of nucleotides such as RNA and DNA, as well as modified forms thereof, peptide nucleic acids (PNAs), locked nucleic acids (LNA™), and the like. In certain applications, the nucleic acid can be a polymer that includes multiple monomer types, e.g., both RNA and DNA subunits.

A nucleic acid is typically single-stranded or double-stranded and will generally contain phosphodiester bonds, although in some cases, as outlined herein, nucleic acid analogs are included that may have alternate backbones, including, for example and without limitation, phosphoramide (Beaucage et al. (1993) Tetrahedron 49(10):1925 and the references therein; Letsinger (1970) J. Org. Chem. 35:3800; Sprinzl et al. (1977) Eur. J. Biochem. 81:579; Letsinger et al. (1986) Nucl. Acids Res. 14: 3487; Sawai et al. (1984) Chem. Lett. 805; Letsinger et al. (1988) J. Am. Chem. Soc. 110:4470; and Pauwels et al. (1986) Chemica Scripta 26:1419), phosphorothioate (Mag et al. (1991) Nucleic Acids Res. 19:1437 and U.S. Pat. No. 5,644,048), phosphorodithioate (Briu et al. (1989) J. Am. Chem. Soc. 111:2321), O-methylphophoroamidite linkages (Eckstein, Oligonucleotides and Analogues: A Practical Approach, Oxford University Press (1992)), and peptide nucleic acid backbones and linkages (Egholm (1992) J. Am. Chem. Soc. 114:1895; Meier et al. (1992) Chem. Int. Ed. Engl. 31:1008; Nielsen (1993) Nature 365:566; and Carlsson et al. (1996) Nature 380:207), which references are each incorporated by reference. Other analog nucleic acids include those with positively charged backbones (Denpcy et al. (1995) Proc. Natl. Acad. Sci. USA 92:6097); non-ionic backbones (U.S. Pat. Nos. 5,386,023, 5,637,684, 5,602,240, 5,216,141 and 4,469,863; Angew (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghvi and P. Dan Cook; Mesmaeker et al. (1994) Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghvi and P. Dan Cook, which references are each incorporated by reference. Nucleic acids containing one or more carbocyclic sugars are also included within the definition of nucleic acids (Jenkins et al. (1995) Chem. Soc. Rev. pp 169-176, which is incorporated by reference). Several nucleic acid analogs are also described in, e.g., Rawls, C & E News Jun. 2, 1997 page 35, which is incorporated by reference. These modifications of the ribose-phosphate backbone may be done to facilitate the addition of additional moieties such as labeling moieties, or to alter the stability and half-life of such molecules in physiological environments.

In addition to naturally occurring heterocyclic bases that are typically found in nucleic acids (e.g., adenine, guanine, thymine, cytosine, and uracil), nucleic acid analogs also include those having non-naturally occurring heterocyclic or other modified bases, many of which are described, or otherwise referred to, herein. In particular, many non-naturally occurring bases are described further in, e.g., Seela et al. (1991) Hely. Chim. Acta 74:1790, Grein et al. (1994) Bioorg. Med. Chem. Lett. 4:971-976, and Seela et al. (1999) Hely. Chim. Acta 82:1640, which are each incorporated by reference. To further illustrate, certain bases used in nucleotides that act as melting temperature (Tm) modifiers are optionally included. For example, some of these include 7-deazapurines (e.g., 7-deazaguanine, 7-deazaadenine, etc.), pyrazolo[3,4-d]pyrimidines, propynyl-dN (e.g., propynyl-dU, propynyl-dC, etc.), and the like. See, e.g., U.S. Pat. No. 5,990,303, entitled “SYNTHESIS OF 7-DEAZA-2′-DEOXYGUANOSINE NUCLEOTIDES,” which issued Nov. 23, 1999 to Seela, which is incorporated by reference. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytosine; 5-fluorocytosine; 5-chlorocytosine; 5-iodocytosine; 5-bromocytosine; 5-methylcytosine; 5-propynylcytosine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, and the like.

“Accessibility” of a DNA region to a DNA modifying agent, as used herein, refers to the ability of a particular DNA region in a chromosome of a cell to be contacted and modified by a particular DNA modifying agent. Without intending to limit the scope of the invention, it is believed that the particular chromatin structure comprising the DNA region will affect the ability of a DNA modifying agent to modify the particular DNA region. For example, the DNA region may be wrapped around histone proteins and further may have additional nucleosomal structure that prevents, or reduces access of, the DNA modifying agent to the DNA region of interest.

A “Type II-S restriction enzyme” is used with its usual meaning the art and refers to a restriction enzyme that recognizes a particular recognition sequence in DNA and then cleaves the DNA molecule outside of that recognition sequence. Exemplary Type II-S restriction enzymes include, but are not limited to, MnII, FokI, and AlwI.

### BRIEF DESCRIPTION OF THE DRAWINGS

FIG. 1 illustrates a schematic representation of the chromatin. Eukaryotic DNA can be classified into two general states, euchromatin, where the DNA is loosely packaged, accessible and transcriptionally competent and heterochromatin, where the DNA is tightly packaged, inaccessible and transcriptionally silent. Epigenetics controls the transition between these two states. The assay described herein can assess chromatin structure, the functional consequence of epigenetic events.

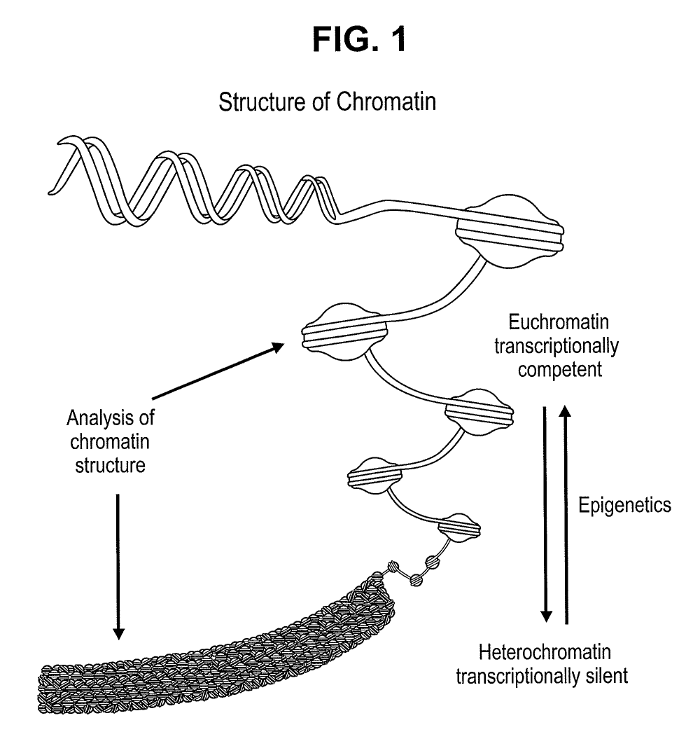

FIG. 2 illustrates a schematic representation of the assay. The culture media is aspirated and a permeabilization/digestion buffer is added. The nuclease diffuses into the cell, enters the nucleus and digests accessible chromatin, but inaccessible chromatin (represented as a thick line towards the bottom of the Figure) is not digested.

FIG. 3 illustrates a schematic representation of an exemplary workflow.

FIG. 4 illustrates an analytic approach to assess the data generated when Mnl 1 is used as a probe. Two DNA samples are analyzed in each assay, one DNA sample is a control that is isolated from cells that are treated with buffer but no nuclease, the other sample is isolated from cells treated with buffer and nuclease. Both samples are analyzed by real-time PCR with the total primer set and the intact primer set. (A) ΔCt (uncut) is calculated by subtracting intact Ct from total Ct using the control DNA sample data. (B) ΔCt (cut) is calculated by subtracting intact Ct from total Ct using the nuclease-treated DNA sample data. Genes that are accessible are cut at the MnlI site, have less intact DNA and a larger ΔCt (cut) that is in proportion to the extent of digestion and reflects greater chromatin accessibility. The amount of inaccessible or “locked-down” DNA is calculated by the formula 2̂(ΔCt (uncut)−ΔCt (cut)).

FIG. 5 is a diagram of the target genes analyzed. The gray area represents the transcribed region. The location of Mnl 1 sites and the TATA box are indicated. GAPDH is a housekeeping gene that is expressed in most cell lines and is expected to be in an accessible chromatin structure. Hemoglobin beta chain (HBB) is silenced in most cells and is expected to be in an inaccessible chromatin structure. Glutathione-s-transferase pi (GSTP1) is epigenetically inactivated in prostate cancer it is expressed in non-cancerous RWPE-1 prostate cells, but is silenced in cancerous LNCaP prostate cells.

FIG. 6 illustrates the target genes analyzed and the location of primer sets. All genes are analyzed with a total primer set that does not cross any MnlI I sites and will amplify all DNA strands, as well as an intact primer set that crosses two to four MalI sites and will only amplify DNA strands that are not cut at these sites.

FIG. 7 provides a summary of analysis of gDNA integrity using agarose gel electrophoresis. Hela cells were treated with permeabilization/digestion buffer that lacked nuclease (M, MnlI; D, DNase I), permeabilization/digestion buffer that lacked lysolecithin, or permeabilization/digestion buffer that contains both nuclease and lysolecithin. gDNA was isolated and analyzed by agarose gel electrophoresis. The results indicate that both nuclease and lysolecithin are required for significant gDNA digestion.

FIG. 8 illustrates fluorescence as a function of cycle number for HBB in MnlI treated Hela cells. Hela cells were treated as indicated. gDNA was isolated and analyzed with primers that amplify the HBB gene. The Ct values of the complete reactions (+, ×) are similar to the control reactions when using the HBB intact primer set. This implies that HBB is in an inaccessible chromatin configuration in Hela cells.

FIG. 9 illustrates fluorescence as a function of cycle number for GAPDH in MnlI treated Hela cells. Hela cells were treated as indicated, gDNA was isolated and analyzed with primers that amplify the GAPDH gene. The Ct values of the complete reactions (+, ×) are higher than the control reactions (the curves are shifted right) when using the GAPDH intact primer set. This implies that GAPDH is in an accessible chromatin configuration in Hela cells.

FIG. 10 illustrates fluorescence as a function of cycle number for MnlI treated Hela cells. Hela cells were treated as indicated, gDNA was isolated and analyzed with primers that amplify the GSTP1 gene. The Ct values of the complete reactions (+, ×) are higher than the control reactions (the curves are shifted right) when using the GSTP1 intact primer set. This implies that GSTP1 is in an accessible chromatin configuration in Hela cells.

FIG. 11 illustrates the data of the three DNA regions (HBB, GAPDH, and GSTP1) in table form, with output calculated as percentage of total DNA that was inaccessible.

FIG. 12 illustrates fluorescence as a function of cycle number for HBB in MnlI-treated prostate cells. In both cell lines, the Ct values of the + enzyme reaction using the HBB intact primer set (×) is similar to the + enzyme reaction using the HBB total primer set (∘). This implies that HBB is in an inaccessible chromatin configuration in RWPE-1 and LNCaP cells.

FIG. 13 illustrates fluorescence as a function of cycle number for GAPDH in MnlI-treated prostate cells. In both cell lines, the Ct values of the + enzyme reaction using the GAPDH intact primer set (×) are higher (the curves are shifted right) than the + enzyme reaction using the GAPDH total primer set (∘). This implies that GAPDH is in an accessible chromatin configuration in RWPE-1 and LNCaP cells.

FIG. 14 illustrates fluorescence as a function of cycle number for GSTP1 in MnlI-treated prostate cells. In RWPE-1 cells the Ct value of the + enzyme reaction using the GSTP1 intact primer set (×) is higher (the curves are shifted right) than the + enzyme reaction using the GSTP1 total primer set (∘). This implies that GSTP1 is in an accessible chromatin configuration in RWPE-1 cells. In contrast, in LNCaP cells the Ct values of the + enzyme reaction using the GSTP1 intact primer set (×) is similar to the + enzyme reaction using the GSTP1 total primer set (∘). This implies that GSTP1 is in an inaccessible chromatin configuration in LNCaP cells.

FIG. 15 illustrates a summary of data from prostate cells. The percentage of inaccessible DNA was calculated as described in the materials and methods section. The results indicate that in both cell lines HBB is in an inaccessible chromatin structure and GAPDH is in an accessible chromatin structure. GSTP1 is in an accessible chromatin in RWPE-1 cells and in an inaccessible chromatin structure in LNCaP cells.

FIG. 16 illustrates an analytic approach to assess the data generated when DNase I is used as a chromatin structure probe. Two DNA samples are analyzed in each assay, one DNA sample is a control that is isolated from cells that are treated with buffer but no nuclease, the other sample is isolated from cells treated with buffer and nuclease. Both samples are analyzed by real-time PCR with a primer set that amplifies an inaccessible reference gene (HBB). ΔCt (ref) represents the extent of nuclease digestion of inaccessible chromatin and is calculated by subtracting the Ct associated with the no nuclease curve from the Ct associated with the + nuclease curve. ΔCt (target) represents the extent of nuclease digestion of the target chromatin region and is calculated by subtracting the Ct associated with the no nuclease curve from the Ct associated with the + nuclease curve. The amount of inaccessible or “locked-down” DNA associated with the target gene is calculated by the formula 2̂(ΔCt (ref)−ΔCt (target)).

FIG. 17 is a diagram of the target genes analyzed and the location of primer sets. The gray area represents the transcribed region. The black regions with arrows represent the location and direction of each primers.

FIG. 18 illustrates fluorescence as a function of cycle number for HBB in DNase I-treated cell lines. In all cell lines, the Ct values of the + nuclease reactions (grey lines) is similar to the no nuclease reactions (black lines). This implies that HBB is in an inaccessible chromatin configuration in all cell lines.

FIG. 19 illustrates fluorescence as a function of cycle number for GAPDH in DNase I-treated cell lines. In all cell lines, the Ct values of the + nuclease reactions (grey lines) are significantly higher than the Ct values of the no nuclease reactions (black lines). This implies that GAPDH is in an accessible chromatin configuration in all cell lines.

FIG. 20 illustrates fluorescence as a function of cycle number for GSTP1 in DNase I-treated cell lines. We observe a cell-line dependent difference in the ΔCt (GSTP1) value. In Hela and HCT15 cells ΔCt (GSTP1) is large indicating that GSTP1 is in an accessible configuration in these cell lines. In LNCaP cells ΔCt (GSTP1) is small indicating that GSTP1 is in inaccessible chromatin. In PC3 cells ΔCt (GSTP1) has an intermediate value indicating that GSTP1 is in a partially accessible chromatin structure. These results are highly consistent with the level of GSTP1 mRNA expression detected in the different cell lines (see FIG. 21).

FIG. 21 illustrates a summary of data from the experiments using DNase I as a chromatin structure probe. The percentage of inaccessible DNA was calculated as described in FIG. 16. The relative level of GAPDH and GSTP1 RNA expression was calculated by analyzing mRNA isolated from the different cell lines by qRT-PCR. The results indicate that target gene chromatin structure correlates well with the level of target gene expression. This implies that the assay described is a useful tool to assess epigenetic effects on gene regulation.

### DETAILED DESCRIPTION

### I. Introduction

The invention allows for analysis of chromatin structure by permeabilizing or disrupting cell membranes and modifying genomic DNA in cells and then quantifying the extent of modification in various loci. The extent of modification at a particular locus reflects the accessibility of that portion of the chromosome to the modifying agent, and thus reflects the state of the chromatin.

One advantage of the present invention is the discovery that one can permeabilize and modify intact chromatin in cell simultaneously, e.g., by contacting cells with one buffer that includes a permeabilization agent and a DNA modifying or cleaving agent. This allows for extremely rapid generation of results. Further, this method eliminates cumbersome and potentially artifact-inducing steps such as isolation of nuclei, etc., as required in some previous methods of chromatin analysis.

### II. General Method

The methods of the invention involve simultaneous permeabilization of a cell and contacting the cell with a DNA modifying agent under conditions such that genomic DNA in the cell has varying accessibility to the modifying agent (due to differences in chromatin structure) and then quantifying the amount of modification in a DNA region. The varying accessibility of the DNA can reflect nucleosomal structure of the genomic DNA. For example, in some embodiments, DNA regions that are more accessible to DNA modifying agents are likely in more “loose” chromatin structures.

A variety of eukaryotic cells can be used in the present invention. In some embodiments, the cells are animal cells, including but not limited to, human, or non-human, mammalian cells. Non-human mammalian cells include but are not limited to, primate cells, mouse cells, rat cells, porcine cells, and bovine cells. In some embodiments, the cells are plant cells. Cells can be, for example, cultured primary cells, immortalized culture cells or can be from a biopsy or tissue sample, optionally cultured and stimulated to divide before assayed. Cultured cells can be in suspension or adherent prior to and/or during the permeabilization and/or DNA modification steps. Cells can be from animal tissues, biopsies, etc. For example, the cells can be from a tumor biopsy.

The present methods can include correlating accessibility of a DNA region to transcription from that same region. In some embodiments, experiments are performed to determine a correlation between accessibility and gene expression and subsequently accessibility of a DNA modifying agent to a particular DNA region can be used to predict transcription from the DNA region. In some embodiments, transcription from a DNA region and accessibility of that region to DNA modifying agents are both determined. A wide variety of methods for measuring transcription are known and include but are not limited to, the use of northern blots and RT-PCR.

In some embodiments, the DNA methylation status of a region can be correlated with accessibility of a DNA region to the DNA modifying agent. In some embodiments, experiments are performed to determine a correlation between accessibility and DNA methylation in the region and subsequently accessibility of a DNA modifying agent to a particular DNA region can be used to predict DNA methylation from the DNA region. In some embodiments, methylation of a DNA region and accessibility of that region to DNA modifying agents are both determined. A wide variety of methods for measuring DNA methylation are known and include but are not limited to, the use of bisulfite (e.g., in sequencing and/or in combination with methylation-sensitive restriction enzymes (see, e.g., Eads et al., Nucleic Acids Research 28(8): E32 (2002)) and the high resolution melt assay (HRM) (see, e.g., Wodjacz et al, Nucleic Acids Research 35(6):e41 (2007)).

The invention provides for, following permeabilization/DNA modification, comparisons of quantity or other physical characteristic of a first DNA region with a second DNA region in a cell's genome. Alternatively, or in addition, one can compare quantity or other physical characteristic of the first DNA region in two different cells. For example, the two cells can represent diseased and healthy cells or tissues, different cell types, different stages of development (including but not limited to stem cells or progenitor cells), etc. Thus, by using the methods of the invention one can detect differences in chromatin structure between cells and/or determine relative chromatin structures between two or more DNA regions (e.g., genes) within one cell. In addition, one can determine the effect of a drug, chemical or environmental stimulus on the chromatin structure of a particular region in the same cells or in different cells.

### III. Permeabilizing and Disrupting Cells

Cell membranes can be permeabilized or disrupted in any way known in the art. As explained herein, the present methods involve contacting the genomic DNA prior to isolation of the DNA and thus methods of permeabilizing or disrupting the cell membrane will not disrupt the structure of the genomic DNA of the cell such that nucleosomal or chromatin structure is destroyed.

In some embodiments, the cell membrane is contacted with an agent that permeabilizes or disrupts the cell membrane. Lysolipids are an exemplary class of agents that permeabilize cell membranes. Exemplary lysolipids include, but are not limited to, lysophosphatidylcholine (also known in the art as lysolecithin) or monopalmitoylphosphatidylcholine. A variety of lysolipids are also described in, e.g., WO/2003/052095.

Non ionic detergents are an exemplary class of agents that disrupt cell membranes. Exemplary nonionic detergents, include but are not limited to, NP40, Tween 20 and Triton X-100.

One advantage of the present invention is the simultaneous delivery of the permeabilization agent and the DNA cleaving or DNA modifying agent. Thus, in some embodiments, a buffer comprising both agents is contacted to the cell. The buffer should be adapted for maintaining activity of both agents while maintaining the structure of the cellular chromatin.

Alternatively, electroporation or biolistic methods can be used to permeabilize a cell membrane such that a DNA modifying agent is introduced into the cell and can thus contact the genomic DNA. A wide variety of electroporation methods are well known and can be adapted for delivery of DNA modifying agents as described herein. Exemplary electroporation methods include, but are not limited to, those described in WO/2000/062855. Biolistic methods include but are not limited to those described in U.S. Pat. No. 5,179,022.

### IV. DNA Modifying Agents

Following permeabilization, or simultaneously with permeabilization (e.g., during electroporation or during incubation with permeabilizing agent), a DNA modifying agent is introduced such that the agent contacts the genomic DNA, thereby introducing modifications into the DNA. A wide variety of DNA modifying agents can be used according to the present invention.

In some embodiments, the DNA modifying agents are contacted to the permeabilized cells following removal of the permeabilizing agent, optionally with a change of the buffer. Alternatively, in some preferred embodiments, the DNA modifying agent is contacted to the genomic DNA without one or more intervening steps (e.g., without an exchange of buffers, washing of the cells, etc.). As noted above, this latter approach can be convenient for reducing the amount of labor and time necessary and also removes a potential source of error and contamination in the assay.

The quantity of DNA modifying agent used, as well as the time of the reaction with the DNA modifying agent will depend on the agent used. Those of skill in the art will appreciate how to adjust conditions depending on the agent used. Generally, the conditions of the DNA modifying step are adjusted such that a “complete” digestion is not achieved. Thus, for example, in some embodiments, the conditions of the modifying step is set such that the positive control—i.e., the control where modification is accessible and occurs—occurs at a high level but less than 100%, e.g., between 80-95%, 80-99%, 85-95%, 90-98%, etc.

A. Restriction Enzymes

In some embodiments, the DNA modifying agent is a restriction enzyme. Thus, in these embodiments, the modification introduced into the genomic DNA is a sequence-specific single-stranded (e.g., a nick) or double-stranded cleavage event. A wide variety of restriction enzymes are known and can be used in the present invention.

Any type of restriction enzyme can be used. Type I enzymes cut DNA at random far from their recognition sequences. Type II enzymes cut DNA at defined positions close to or within their recognition sequences. Some Type II enzymes cleave DNA within their recognition sequences. Type II-S enzymes cleave outside of their recognition sequence to one side. The third major kind of type II enzyme, more properly referred to as “type IV,” cleave outside of their recognition sequences. For example, those that recognize continuous sequences (e.g., AcuI: CTGAAG) cleave on just one side; those that recognize discontinuous sequences (e.g., BcgI: CGANNNNNNTGC) cleave on both sides releasing a small fragment containing the recognition sequence. Type III cleave outside of their recognition sequences and require two such sequences in opposite orientations within the same DNA molecule to accomplish cleavage.

The methods of the invention can be adapted for use with any type of restriction enzyme or other DNA cleaving enzyme. In some embodiments, the enzyme is one or more that cleaves relatively close (e.g., within 5, 10, or 20 base pairs) of the recognition sequence. Such enzymes can be of particular use in assaying chromatin structure as the span of DNA that must be accessible to achieve cutting is larger than the recognition sequence itself and thus may involve a wider span of DNA that is not in a “tight” chromatin structure. Sequence-specific restriction enzymes can provide improved quantitative results in part because controls based on the same DNA region can be designed as described herein (e.g., in the Examples). Thus, the number of total and digested copies can be more accurately determined compared to, e.g., digestion with sequence non-specific endonucleases (“DNases”). Unlike DNase I, cleavage by TypeII-S restriction enzymes can be sensitive to the binding of histones to the DNA region(s) of interest. Exemplary enzymes that cut outside their recognition sequence includes, e.g., Type II-S, Type III, and Type IV enzymes. Type II-S restriction enzymes, include but are not limited to, MnII, FokI and AlwI.

In some embodiments, more than one (e.g., two, three, four, etc.) restriction enzymes are used. Combinations of enzymes can involve combinations of enzymes all from one type or can be mixes of different types.

Intact or cut DNA can subsequently be separately detected and quantified and the number of intact and/or cut copies of a DNA region can be determined as described herein.

In some embodiments, the permeabilizing or membrane disrupting agent is added prior to the restriction enzyme. In some embodiments, the restriction enzyme and permeabilizing or disrupting agent are added simultaneously (e.g., in or with appropriate buffers). Even if both agents are not initially contacted to a cell at the same moment, one can still achieve simultaneous permeabilization and contact with a DNA modifying agent because permeabilization can be an ongoing process. Thus, for example, addition of a permeabilizing agent followed soon after (before permeabilization is substantially complete) with a DNA modifying agent can be considered “simultaneously” permeabilizing and contacting the cell with the DNA modifying agent. “Simultaneous” means no intervening manipulations occur (including but not limited to change of buffer, centrifugation, etc.) between addition of the permeabilization and modifying agent.

In some embodiments, 0.5% lysolecithin (w/v), 50 mM NaCl, 10 mM Tris-HCl pH 7.4, 10 mM MgCl2, 1 mM DTT, 100 ug/ml BSA and 0-500 units/ml MnlI (or other restriction enzyme) are used. In some embodiments, 0.25% lysolecithin (w/v), 50 mM NaCl, 10 mM Tris-HCl pH 7.4, 10 mM MgCl2, 1 mM DTT, 100 ug/ml BSA and 0-500 units/ml MnlI (or other restriction enzyme) are used. In some embodiments, 0.75% lysolecithin (w/v), 50 mM NaCl, 10 mM Tris-HCl pH 7.4, 10 mM MgCl2, 1 mM DTT, 100 ug/ml BSA and 0-500 units/ml MnlI (or other restriction enzyme) are used. In some embodiments, 1% lysolecithin (w/v), 50 mM NaCl, 10 mM Tris-HCl pH 7.4, 10 mM MgCl2, 1 mM DTT, 100 ug/ml BSA and 0-800 units/ml MnlI (or other restriction enzyme) are used.

Following permeabilization and digestion, the digestion optionally is stopped and the cells are lysed, optionally by simultaneous addition of a lysis/stop buffer and/or increased temperature. Exemplary lysis/stop buffers can include sufficient chelator and detergent to stop the reaction and to lyse the cells. For example, in some embodiments, the lysis/stop buffer comprises 100 mM Tris-HCl pH 8, 100 mM NaCl, 100 mM EDTA, 5% SDS (w/v) and 3 mg/ml proteinase K. In some embodiments, the lysis/stop buffer comprises 100 mM Tris-HCl pH 8, 100 mM NaCl, 100 mM EDTA, 1% SDS (w/v) and 3 mg/ml proteinase K. In some embodiments, the lysis/stop buffer comprises 200 mM Tris-HCl pH 8, 100 mM NaCl, 500 mM EDTA, 5% SDS (w/v) and 5 mg/ml proteinase K.

B. DNases

In some embodiments, an enzyme that cuts or nicks DNA in a sequence non-specific manner is used as a DNA modifying agent. Thus, in some embodiments, the DNA modifying agent is a sequence non-specific endonuclease (also referred to herein as a “DNase”).

Any sequence non-specific endonuclease (e.g., any of DNase I, II, III, IV, V, VI, VII) can be used according to the present invention. For example, any DNase, including but not limited to, DNase I can be used. DNases used can include naturally occurring DNases as well as modified DNases. An example of a modified DNase is TURBO DNase (Ambion), which includes mutations that allow for “hyperactivity” and salt tolerance. Exemplary DNases, include but are not limited, to Bovine Pancreatic DNase I (available from, e.g., New England Biolabs.

Intact DNA can subsequently be separately detected and quantified and the number of intact and/or cut copies of a DNA region can be determined as described herein.

In some embodiments, the permeabilizing or membrane disrupting agent is added prior to the DNase. In some embodiments, the DNase and permeabilizing or disrupting agent are added simultaneously (e.g., with appropriate buffers). In some embodiments, the permeabilization/digestion buffer comprises 0.25% lysolecithin (w/v), 10 mM Tris-HCl pH 7.4, 2.5 mM MgCl₂, 0.5 mM CaCl2 and 0-200 units/ml DNase I. In some embodiments, the permeabilization/digestion buffer comprises 0.5% lysolecithin (w/v), 10 mM Tris-HCl pH 7.4, 2.5 mM MgCl₂, 0.5 mM CaCl2 and 0-200 units/ml DNase I. In some embodiments, the permeabilization/digestion buffer comprises 0.75% lysolecithin (w/v), 10 mM Tris-HCl pH 7.4, 2.5 mM MgCl₂, 0.5 mM CaCl2 and 0-500 units/ml DNase I. In some embodiments, the permeabilization/digestion buffer comprises 0.25% lysolecithin (w/v), 10 mM Tris-HCl pH 7.4, 2.5 mM MgCl₂, 0.5 mM CaCl2 and 0-500 units/ml DNase I. Permeabilization and lysis can be stopped, for example, as described above for restriction enzymes.

As discussed elsewhere, use of a DNase or other general DNA cleaving agent can be enhanced by monitoring extent of cleavage between at least two different DNA regions, one being the target, and the other being a DNA region that is generally always accessible or is generally always inaccessible in any of the test conditions. Examples of such genes are discussed elsewhere herein and are known or can be identified. For example, DNA regions encompassing ‘housekeeping” genes are generally always accessible. The relative amount of remaining target compared to the control can then be used to determine relative chromatin structure at the target DNA region.

C. Methytransferases

In some embodiments of the invention, the DNA modifying agent generates a covalent modification to the DNA. For example, in some embodiments, the DNA modifying agents of the invention are methyltransferases.

A variety of methyltransferases are known in the art and can be used in the invention. In some embodiments, the methyltransferase used adds a methyl moiety to adenosine in DNA. Examples of such methyltransferases include, but are not limited to, DAM methyltransferase. Because adenosine is not methylated in eukaryotic cells, the presence of a methylated adenosine in a particular DNA region indicates that a DAM methyltransferase (or other methyltransferase with similar activity) was able to access the DNA region. Adenosine methylation can be detected, for example, using a restriction enzyme whose recognition sequence includes a methylated adenosine. An example of such an enzyme includes, but is not limited to, DpnI. Cutting by the restriction enzyme can then be quantified as described herein (for example, where intact DNA is amplified but cut DNA is not—or using LM-PCR, to amplify cut DNA but not intact DNA.

In some embodiments, the methyltransferase methylates cytosines in GC sequences. Examples of such methyltransferases include but are not limited to MCviPI. See, e.g., Xu et al., Nuc. Acids Res. 26(17): 3961-3966 (1998). Because GC sequences are not methylated in eukaryotic cells, the presence of a methylated GC sequence in a particular DNA region indicates that the DNA modifying agent (i.e., a methyltransferase that methylates cytosines in GC sequences) was able to access the DNA region. Methylated GC sequences can be identified using any number of techniques. In some embodiments, the method for detecting methylated GC sequences comprises bisulfite conversion. Bisulfite conversion involves contacting the DNA with a sufficient amount of bisulfite to convert unmethylated cytosines to uracil. Methylated cytosines are not converted. Thus, DNA regions containing a GC sequence can be contacted with a methyltransferase that methylates cytosines in GC sequences, isolated, and then contacted with bisulfite. If the C in the GC sequence is not methylated, the C will be converted to U (or T if subsequently amplified), whereas a methylated C will remain a C. Any number of methods, including but not limited to, nucleotide sequencing and methods involving primer extension or primer-based amplification and/or methylation-sensitive restriction digests can be used to detect the presence or absence of a bisulfite converted C (e.g., MSnuPE, MSP or Methyllight, high resolution melt analysis; pyrosequencing, etc.). See, e.g., Fraga, et al., Biotechniques 33:632, 634, and 636-649 (2002); El-Maarri O Adv Exp Med Biol 544:197-204 (2003); Laird, Nat Rev Cancer 3:253-266 (2003); and Callinan, Hum Mol Genet 15 Spec No 1:R95-101 (2006).

In some embodiments, the methyltransferase methylates cytosines in CG (also known as “CpG”) sequences. Examples of such methyltransferases include but are not limited to M.SssI. Use of such methyltransferases will generally be limited to use for those DNA regions that are not typically methylated. This is because CG sequences are endogenously methylated in eukaryotic cells and thus it is not generally possible to assume that a CG sequence is methylated by the modifying agent rather than an endogenous methyltransferase except in such DNA regions where methylation is rare. As for GC sequences, methylation of CG sequences can be detected by any number of methods, including methods involving bisulfite conversion.

D. Chemicals

In some embodiments, the DNA modifying agent comprises a DNA modifying chemical. As most DNA modifying chemicals are relatively small compared to chromatin, use of DNA modifying chemicals without a fusion partner may not be effective in some circumstances as there will be little if any difference in the extent of accessibility of different DNA regions. Therefore, in some embodiments, the DNA modifying agent comprises a molecule having steric hindrance linked to a DNA modifying chemical. The molecule having steric hindrance can be any protein or other molecule that results in differential accessibility of the DNA modifying agent depending on chromatin structure. This can be tested, for example, by comparing results to those using a DNase or restriction enzyme as described herein.

In some embodiments, the molecule having steric hindrance will be at least 5, 7, 10, or 15 kD in size. Those of skill in the art will likely find it convenient to use a polypeptide as the molecule with steric hindrance. Any polypeptide can be used that does not significantly interfere with the DNA modifying agent's ability to modify DNA. In some embodiments, the polypeptide is a double-stranded sequence-non-specific nucleic acid binding domain as discussed in further detail below.

The DNA modifying chemicals of the present invention can be linked directly to the molecule having steric hindrance or via a linker. A variety of homo and hetero bifunctional linkers are known and can be used for this purpose.

Exemplary DNA modifying chemicals include but are not limited to hydrazine (and derivatives thereof, e.g., as described in Mathison et al., Toxicology and Applied Pharmacology 127(1):91-98 (1994)) and dimethyl sulfate. In some embodiments, hydrazine introduces a methyl groups to guanosines in DNA or otherwise damages DNA. In some embodiments, dimethyl sulfate methylates guanine or results in the base-specific cleavage of guanine in DNA by rupturing the imidazole rings present in guanine.

Detection of modifications by DNA modifying chemical will depend on the type of DNA modification that occurs. In some embodiments, to detect dimethyl sulfate or hydrazine modification the DNA is treated with piperidine at high temperature (90° C.). The DNA breaks at the site of DNA modification and the breaks can be detected in the same ways as nuclease cutting is detected as described herein.

E. DNA Binding Domains to Improve DNA Modifying Agents

In some embodiments, the DNA modifying or cleavage agents of the invention are fused or otherwise linked to a double-stranded sequence-non-specific nucleic acid binding domain (e.g., a DNA binding domain). In cases where the DNA modifying agent is a polypeptide, the double-stranded sequence-non-specific nucleic acid binding domain can be synthesized, for example, as a protein fusion with the DNA modifying agent via recombinant DNA technology. A double-stranded sequence-non-specific nucleic acid binding domain is a protein or defined region of a protein that binds to double-stranded nucleic acid in a sequence-independent manner, i.e., binding does not exhibit a gross preference for a particular sequence. In some embodiments, double-stranded nucleic acid binding proteins exhibit a 10-fold or higher affinity for double-stranded versus single-stranded nucleic acids. The double-stranded nucleic acid binding proteins in some embodiments of the invention are thermostable. Examples of such proteins include, but are not limited to, the Archaeal small basic DNA binding proteins Sac7d and Sso7d (see, e.g., Choli et al., Biochimica et Biophysica Acta 950:193-203, 1988; Baumann et al., Structural Biol. 1:808-819, 1994; and Gao et al, Nature Struc. Biol. 5:782-786, 1998), Archael HMf-like proteins (see, e.g., Starich et al., J. Molec. Biol. 255:187-203, 1996; Sandman et al., Gene 150:207-208, 1994), and PCNA homologs (see, e.g., Cann et al., J. Bacteriology 181:6591-6599, 1999; Shamoo and Steitz, Cell:99, 155-166, 1999; De Felice et al., J. Molec. Biol. 291, 47-57, 1999; and Zhang et al., Biochemistry 34:10703-10712, 1995). See also European Patent 1283875B1 for addition information regarding DNA binding domains.

Sso7d and Sac7d

Sso7d and Sac7d are small (about 7,000 kd MW), basic chromosomal proteins from the hyperthermophilic archaeabacteria Sulfolobus solfataricus and S. acidocaldarius, respectively. These proteins are lysine-rich and have high thermal, acid and chemical stability. They bind DNA in a sequence-independent manner and when bound, increase the T_{M} of DNA by up to 40° C. under some conditions (McAfee et al., Biochemistry 34:10063-10077, 1995). These proteins and their homologs are typically believed to be involved in stabilizing genomic DNA at elevated temperatures.

HMF-Like Proteins

The HMf-like proteins are archaeal histones that share homology both in amino acid sequences and in structure with eukaryotic H4 histones, which are thought to interact directly with DNA. The HMf family of proteins form stable dimers in solution, and several HMf homologs have been identified from thermostable species (e.g., Methanothermus fervidus and Pyrococcus strain GB-3a). The HMf family of proteins, once joined to Taq DNA polymerase or any DNA modifying enzyme with a low intrinsic processivity, can enhance the ability of the enzyme to slide along the DNA substrate and thus increase its processivity. For example, the dimeric HMf-like protein can be covalently linked to the N terminus of Taq DNA polymerase, e.g., via chemical modification, and thus improve the processivity of the polymerase.

Those of skill in the art will recognize that other double-stranded sequence-non-specific nucleic acid binding domain are known in the art and can also be used as described herein.

F. Isolation of DNA Following the DNA Modifying Step

In some embodiments, following the DNA modification/cleavage step, genomic DNA is isolated from the cells according to any method available. Essentially any DNA purification procedure can be used so long as it results in DNA of acceptable purity for the subsequent quantification step(s). For example, standard cell lysis reagents can be used to lyse cells. Optionally a protease (including but not limited to proteinase K) can be used. DNA can be isolated from the mixture as is known in the art. In some embodiments, phenol/chloroform extractions are used and the DNA can be subsequently precipitated (e.g., by ethanol) and purified. In some embodiments, RNA is removed or degraded (e.g., with an RNase or with use of a DNA purification column), if desired.

Optionally, genomic DNA is amplified or otherwise detected directly from the cell lysate without an intermediate purification step.

In some embodiments, intact, modified or unmodified DNA is isolated and cloned into a library. In some cases, one or more specific intact, modified, or unmodified sequence is isolated and/or cloned. Alternatively, a sample having intact, modified, or unmodified DNA regions is used to prepare a library enriched for such regions. Intact DNA, following contact with a DNA cleavage agent, represents DNA that was less accessible to the agent. Similarly, unmodified DNA, following contact with a DNA modifying agent, represents less accessible DNA. Conversely, modified DNA represents DNA that was more accessible to the modifying agent. In some of the above embodiments, intact DNA is purified (e.g., separated) from cleaved DNA and/or modified DNA is purified from unmodified DNA prior to cloning, thereby enriching the cloning pool for one class of DNA. Enriching for modified/unmodified DNA will vary depending on the nature of the modification. In some embodiments, an affinity agent that specifically binds to modified (or unmodified DNA) is used to separate modified from unmodified DNA.

In some embodiments, subtractive libraries are generated. For example, libraries can be generated that are enriched for a diseased cell DNA regions that are intact, modified, or unmodified in the methods of the invention and subsequently subtracted with a corresponding library from a healthy cell, thereby generating a library of differential DNA sequences that are both intact, modified, or unmodified and are specific for the particular disease. Any diseased cell can be used, including but not limited to, cancer cells. Alternate subtractive strategies can also be employed, e.g., between different cell types, cell stages, drug treatments, etc.

G. Detecting Physical Characteristics of the DNA

Any number of physical characteristics of DNA can be detected following contact of the cell with a DNA modifying or DNA cleaving agent. Physical characteristics include, but are not limited to, DNA methylation, melting temperature, GC content, nucleotide sequence, and ability to hybridize to a polynucleotide. A variety of methods are known for detecting such characteristics and can be employed. In some embodiments, following the DNA modification/cleavage step, the physical characteristic determined does not involve DNA footprinting (e.g., the ability of a specific protein or proteins to a specific region of DNA) is not determined. For example, in a non-limiting embodiment, quantification of intact DNA, e.g., using qPCR, does not involve DNA footprinting.

In some embodiments, the physical characteristic is DNA methylation. For example, once relatively accessible DNA has been cleaved by a DNA cleaving agent, one can isolate the remaining intact DNA (representing less accessible DNA) and can then be analyzed for methylation status. A large variety of DNA methylation detection methods are known. In some embodiments, following contact with the DNA modifying or cleavage agent, the DNA is contacted with bisulfite, thereby converting unmethylated cytosines to uracils in the DNA. The methylation of a particular DNA region can then be determined by any number of methylation detection methods, including those discussed herein. In some embodiments, a high resolution melt assay (HRM) is employed to detect methylation status following bisulfite conversion. In this method, a DNA region is amplified following bisulfite conversion and the resulting amplicon's melting temperature is determined. Because the melting temperature will differ depending on whether the cytosines were converted by bisulfite (and subsequently copied as “T's” in the amplification reaction), melting temperature of the amplicon can be correlated to methylation content.

### V. Target DNA Regions

A DNA region is a target sequence of interest within genomic DNA. Any DNA sequence in genomic DNA of a cell can be evaluated for DNA modifying agent accessibility as described herein. DNA regions can be screened to identify a DNA region of interest that displays different accessibility in different cell types, between untreated cells and cells exposed to a drug, chemical or environmental stimulus, or between normal and diseased tissue, for example. Thus, in some embodiments, the methods of the invention are used to identify a DNA region whose change in accessibility acts as a marker for disease (or lack thereof). Exemplary diseases include but are not limited to cancers. A number of genes have been described that have altered DNA methylation and/or chromatin structure in cancer cells compared to non-cancer cells.

In some embodiments, the DNA region is known to be differentially accessible depending on the disease or developmental state of a particular cell. In these embodiments, the methods of the present invention can be used as a diagnostic or prognostic tool. Once a diagnosis or prognosis is established using the methods of the invention, a regimen of treatment can be established or an existing regimen of treatment can be altered in view of the diagnosis or prognosis. For instance, detection of a cancer cell according to the methods of the invention can lead to the administration of chemotherapeutic agents and/or radiation to an individual from whom the cancer cell was detected.

A variety of DNA regions can be detected either for research purposes and/or as a control DNA region to confirm that the reagents were performing as expected. For example, in some embodiments, a DNA region is assayed that is accessible in essentially all cells of an animal. Such DNA regions are useful, for example, as positive controls for accessibility. Such DNA regions can be found, for example, within or adjacent to genes that are constitutive or nearly constitutive. Such genes include those generally referred to as “housekeeping” genes, i.e., genes whose expression are required to maintain basic cellular function. Examples of such genes include, but are not limited to glyceraldehyde-3-phosphate dehydrogenase (GAPDH) and beta actin (ACTB). DNA regions can include all or a portion of such genes, optionally including at least a portion of the promoter.

In some embodiments, a DNA region comprises at least a portion of DNA that is inaccessible in most cells of an animal. Such DNA regions are useful, for example, as negative controls for accessibility. “Inaccessible” in this context refers to DNA regions whose copies are modified in no more than around 20% of the copies of the DNA region. Examples of such gene sequences include those generally recognized as “heterochromatic” and include genes that are only expressed in very specific cell types (e.g., expressed in a tissue or organ-specific fashion). Exemplary genes that are generally inaccessible (with the exception of specific cell types) include, but are not limited to, hemoglobin-beta chain (HBB) and immunoglobulin light chain kappa (IGK).

In some embodiments, the DNA region is a gene sequence which has different accessibility depending on the disease state of the cell or otherwise have variable accessibility depending on type of cells or growth environment. For example, some genes are generally inaccessible in non-cancer cells but are accessible in cancer cells. Examples of genes with variable accessibility include, e.g., Glutathione-s-transferase pi (GSTP1).

In some embodiments, a DNA region of the invention is selected from a gene sequence (e.g., a promoter sequence) from one or more of the following genes cadherin 1 type 1 (E-Cadherin), Cytochrome P450-1A1 (CYP1A1), Ras association domain family 1A (RASSF1A), p15, p16, Death associated protein kinase 1 (DAPK), Adenomatous Polyposis Of The Colon (APC), Methylguanine-DNA Methyltransferase (MGMT), Breast Cancer 1 Gene (BRCA1) and hMLH.

In some embodiments, the DNA regions are selected at random, for example, to identify regions that have differential accessibility between different cell types, different conditions, normal vs. diseased cells, etc.

### VI. Quantifying Copies of the Target Locus

The method for quantifying DNA modifications will depend on the type of DNA modification introduced into the genomic DNA. For example, double stranded DNA cleavage events (e.g., as introduced by a restriction enzyme or DNase or introduced following modification, e.g., by a methylation-sensitive or -dependent restriction enzyme following methyltransferase treatment, or following modification by a DNA modifying chemical as described herein) can be conveniently detected using an amplification reaction designed to generate an amplicon that comprises a DNA region of interest. In the case of cleavage events at defined sites, such as when a sequence-specific restriction enzyme is used, primers are designed to generate an amplicon that spans a potential cleavage site. Only intact DNA will be amplified. If one also knows the amount of total DNA, one can calculate the amount of cleaved DNA as the difference between total and intact DNA. The total amount of DNA can be determined according to any method of DNA quantification known in the art. In some embodiments, the amount of total DNA can be conveniently determined by designing a set of primers that amplify the DNA regardless of modification. This can be achieved, for example, by designing primers that do not span a potential cleavage site, either within the same gene region or in another DNA region. In the case of cleavage events at indeterminate sites, such as when a non sequence-specific nuclease, such as DNase I is used, the use of an inaccessible reference gene should be incorporated as an internal control.

As discussed in more detail below, quantitative amplification (including, for example real-time PCR) methods allow for determination of the amount of intact copies of a DNA region, and when used with various controls can be used to determine the relative amount of intact DNA compared to the total number of copies in the cell. The actual or relative number (e.g., relative to the total number of copies or relative to the number of modified or unmodified copies of a second DNA region) of modified or unmodified copies of the DNA region can thus be calculated.

In some embodiments of the invention, the number of modified copies of a DNA region are determined directly. For example, restriction enzyme cleavage can also be detected and quantified, for example, by detecting specific ligation events, for example, that will occur only in the presence of specific sticky or blunt ends. For example, nucleic acid adaptors comprising sticky ends that are complementary to sticky ends generated by a restriction enzyme can be ligated to the cleaved genomic DNA. The number of ligation events can then be detected and quantified (e.g., by a quantitative amplification method).

In some embodiments, ligation mediated PCR (LM-PCR) is employed to quantify the number of cleaved copies of a DNA region. Methods of LM-PCR are known in the art and were initially described in Pfeifer et al., Science 246: 810-813 (1989). LM-PCR can be performed in real-time for quantitative results if desired.

Quantitative amplification methods (e.g., quantitative PCR or quantitative linear amplification) involve amplification of an nucleic acid template, directly or indirectly (e.g., determining a Ct value) determining the amount of amplified DNA, and then calculating the amount of initial template based on the number of cycles of the amplification. Amplification of a DNA locus using reactions is well known (see U.S. Pat. Nos. 4,683,195 and 4,683,202; PCR PROTOCOLS: A GUIDE TO METHODS AND APPLICATIONS (Innis et al., eds, 1990)). Typically, PCR is used to amplify DNA templates. However, alternative methods of amplification have been described and can also be employed, as long as the alternative methods amplify intact DNA to a greater extent than the methods amplify cleaved DNA. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol Biotechnol. 20(2):163-79 (2002). Amplifications can be monitored in “real time.”

In some embodiments, quantitative amplification is based on the monitoring of the signal (e.g., fluorescence of a probe) representing copies of the template in cycles of an amplification (e.g., PCR) reaction. In the initial cycles of the PCR, a very low signal is observed because the quantity of the amplicon formed does not support a measurable signal output from the assay. After the initial cycles, as the amount of formed amplicon increases, the signal intensity increases to a measurable level and reaches a plateau in later cycles when the PCR enters into a non-logarithmic phase. Through a plot of the signal intensity versus the cycle number, the specific cycle at which a measurable signal is obtained from the PCR reaction can be deduced and used to back-calculate the quantity of the target before the start of the PCR. The number of the specific cycles that is determined by this method is typically referred to as the cycle threshold (Ct). Exemplary methods are described in, e.g., Heid et al. Genome Methods 6:986-94 (1996) with reference to hydrolysis probes.

One method for detection of amplification products is the 5′-3′ exonuclease “hydrolysis” PCR assay (also referred to as the TaqMan™ assay) (U.S. Pat. Nos. 5,210,015 and 5,487,972; Holland et al., PNAS USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the “TaqMan™ probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

Another method of detecting amplification products that relies on the use of energy transfer is the “beacon probe” method described by Tyagi and Kramer, Nature Biotech. 14:303-309 (1996), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched. When employed in PCR, the molecular beacon probe, which hybridizes to one of the strands of the PCR product, is in the open conformation and fluorescence is detected, while those that remain unhybridized will not fluoresce (Tyagi and Kramer, Nature Biotechnol. 14: 303-306 (1996)). As a result, the amount of fluorescence will increase as the amount of PCR product increases, and thus may be used as a measure of the progress of the PCR. Those of skill in the art will recognize that other methods of quantitative amplification are also available.

Various other techniques for performing quantitative amplification of a nucleic acids are also known. For example, some methodologies employ one or more probe oligonucleotides that are structured such that a change in fluorescence is generated when the oligonucleotide(s) is hybridized to a target nucleic acid. For example, one such method involves is a dual fluorophore approach that exploits fluorescence resonance energy transfer (FRET), e.g., LightCycler™ hybridization probes, where two oligo probes anneal to the amplicon. The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: Scorpions™ probes (e.g., Whitcombe et al., Nature Biotechnology 17:804-807, 1999, and U.S. Pat. No. 6,326,145), Sunrise™ (or Amplifluor™) probes (e.g., Nazarenko et al., Nuc. Acids Res. 25:2516-2521, 1997, and U.S. Pat. No. 6,117,635), and probes that form a secondary structure that results in reduced signal without a quencher and that emits increased signal when hybridized to a target (e.g., Lux probes™).

In other embodiments, intercalating agents that produce a signal when intercalated in double stranded DNA may be used. Exemplary agents include SYBR GREEN™, SYBR GOLD™, and EVAGREEN™. Since these agents are not template-specific, it is assumed that the signal is generated based on template-specific amplification. This can be confirmed by monitoring signal as a function of temperature because melting point of template sequences will generally be much higher than, for example, primer-dimers, etc.

In some embodiments, the quantity of a DNA region is determined by nucleotide sequencing copies in a sample and then determining the relative or absolute number of copies having the same sequence in a sample.

Quantification of modified (or unmodified) DNA regions according to the method of the invention can be further improved, in some embodiments, by determining the relative amount (e.g., a normalized value such as a ratio or percentage) of modified or unmodified copies of the DNA region compared to the total number of copies of that same region. In some embodiments, the relative amount of modified or unmodified copies of one DNA region is compared to the number of modified or unmodified copies of a second (or more) DNA regions. In some embodiments, when comparing between two or more DNA regions, the relative amount of modified or unmodified copies of each DNA region can be first normalized to the total number of copies of the DNA region. Alternatively, when obtained from the same sample, in some embodiments, one can assume that the total number of copies of each DNA region is roughly the same and therefore, when comparing between two or more DNA regions, the relative amount (e.g., the ratio or percentage) of modified or unmodified copies between each DNA region is determined without first normalizing each value to the total number of copies.

In some embodiments, the actual or relative (e.g., relative to total DNA) amount of modified or unmodified copies is compared to a control value. Control values can be conveniently used, for example, where one wants to know whether the accessibility of a particular DNA region exceeds or is under a particular value. For example, in the situation where a particular DNA region is typically accessible in normal cells, but is inaccessible in diseased cells (or vice versa), one may simply compare the actual or relative number of modified or unmodified copies to a control value (e.g., greater or less than 20% modified or unmodified, greater or less than 80% modified or unmodified, etc.). Alternatively, a control value can represent past or expected data regarding a control DNA region. In these cases, the actual or relative amount of a control DNA region are determined (optionally for a number of times) and the resulting data is used to generate a control value that can be compared with actual or relative number of modified or unmodified copies determined for a DNA region of interest.

The calculations for the methods described herein can involve computer-based calculations and tools. The tools are advantageously provided in the form of computer programs that are executable by a general purpose computer system (referred to herein as a “host computer”) of conventional design. The host computer may be conFigured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### VII. Reaction Mixtures

The present invention also provides for reaction mixtures comprising one or more of the reagents as described herein, optionally with a eukaryotic cell (whose chromatin state is to be determined). In some embodiments, the reaction mixtures comprise, e.g., a DNA modifying agent (e.g., a restriction enzyme, a DNase, a methyltransferase or a DNA modifying chemical) and a cell permeabilizing and/or cell disrupting agent and a eukaryotic cell. Other reagents as described herein (including but not limited to bisulfite) can also be included in the reaction mixture of the invention.

### VIII. Kits

The present invention also provides kits for performing the accessibility assays of the present invention. A kit can optionally include written instructions or electronic instructions (e.g., on a CD-ROM or DVD). Kits of the present invention can include, e.g., a DNA modifying agent and a cell permeabilizing and/or cell disrupting agent. DNA modifying agents can include those described herein in detail, including, e.g., a restriction enzyme, a DNase, a methyltransferase or a DNA modifying chemical. In some embodiments, the DNA modifying agent is a Type II-S restriction enzyme, including but not limited to, MnII, FokI and AlwI. Kits of the invention can comprise the permeabilizing agent and the DNA modifying agent in the same vial/container (and thus in the same buffer). Alternatively, the permeabilizing agent and the DNA modifying agent can be in separate vials/containers.

The kits of the invention can also include one or more control cells and/or nucleic acids. Exemplary control nucleic acids include, e.g., those comprising a gene sequence that is either accessible in essentially all cells of an animal (e.g., a housekeeping gene sequence or promoter thereof) or inaccessible in most cells of an animal. In some embodiments, the kits include one or more sets of primers for amplifying such gene sequences (whether or not the actually gene sequences or cells are included in the kits). For example, in some embodiments, the kits include a DNA modifying agent, and a cell permeabilizing and/or cell disrupting agent, and one or more primer sets for amplifying a control DNA region, and optionally one or more primer sets for amplifying a second DNA region, e.g., a target DNA region. In embodiments where the DNA modifying agent is a restriction enzyme, at least two primer sets per DNA region can be included: One primer set for amplifying a portion of the DNA region that includes at least one (e.g., 1, 2, 3, 4, etc.) potential cleavage sites of the restriction enzyme (e.g., useful for calculating the number of unmodified copies), and at least a second primer set for amplifying a portion of the DNA region that does not include any potential cleavage sites (e.g., useful for calculating the total number of copies).

In some embodiments, the kits of the invention comprise one or more of the following:
(i) a cell membrane permeabilizing or disrupting agent;
(ii) a restriction enzyme, DNase, or other DNA modifying agent;
(iii) a “stop” solution capable of preventing further modification by the modifying agent;
(iv) materials for the extraction and/or purification of nucleic acids (e.g., a spin column for purification of genomic DNA and/or removal of non-DNA components such as components of a “stop” solution);
(vi) reagents for PCR/qPCR amplification of DNA, optionally one mixture containing all components necessary for PCR or for qPCR aside from the template and/or polymerase;
(vii) primer sets for PCR/qPCR amplification of specific target genes.

### EXAMPLES

The following examples are offered to illustrate, but not to limit the claimed invention.

### Example 1

### General Approach

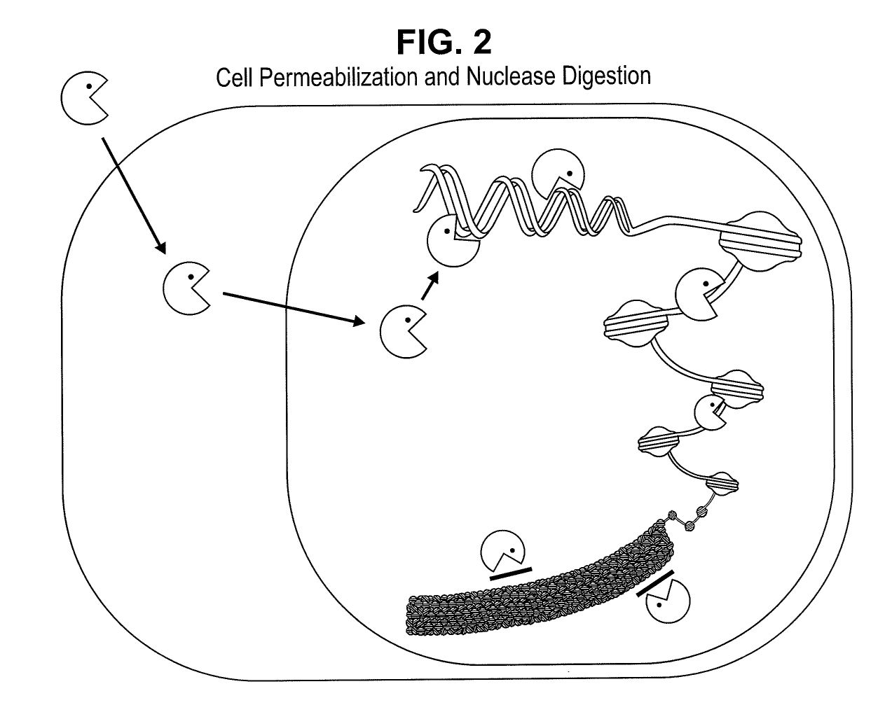

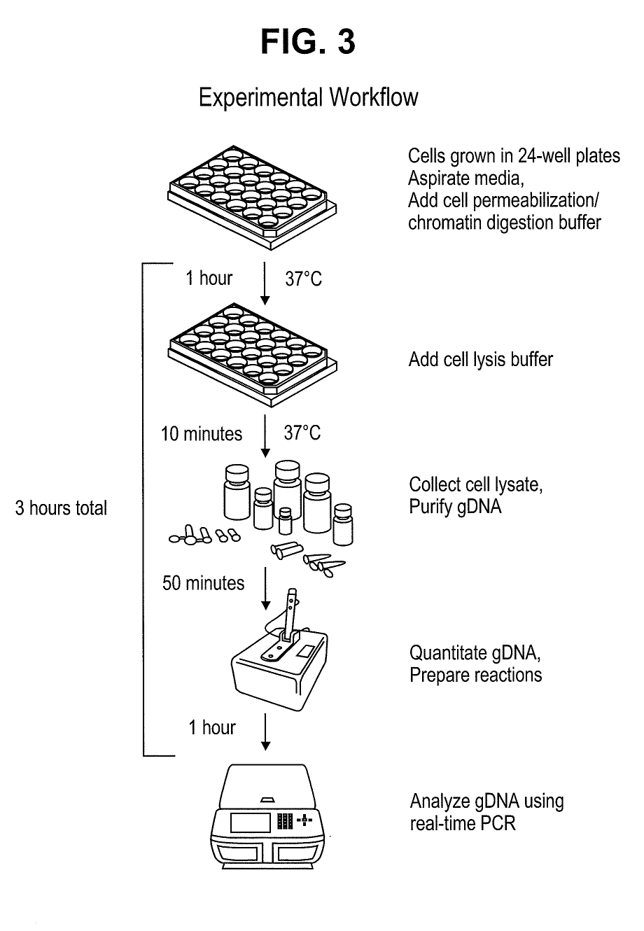

The present assay takes advantage of the difference of accessibility of a DNA modifying agent to different parts of chromatin in a cell. As illustrated in FIG. 2, DNA modifying agents can access certain portions of chromatin more readily than other parts. FIG. 3 illustrates an exemplary workflow of the assay. Adherent cells were grown in 24-well plates as starting material. In this embodiment, two wells are used for each experiment: One well is treated with permeabilization buffer and no nuclease; the other well is treated with permeabilization buffer with nuclease. The entire process, from cells to real-time PCR, takes about three hours and results are available on the day of cell harvest.

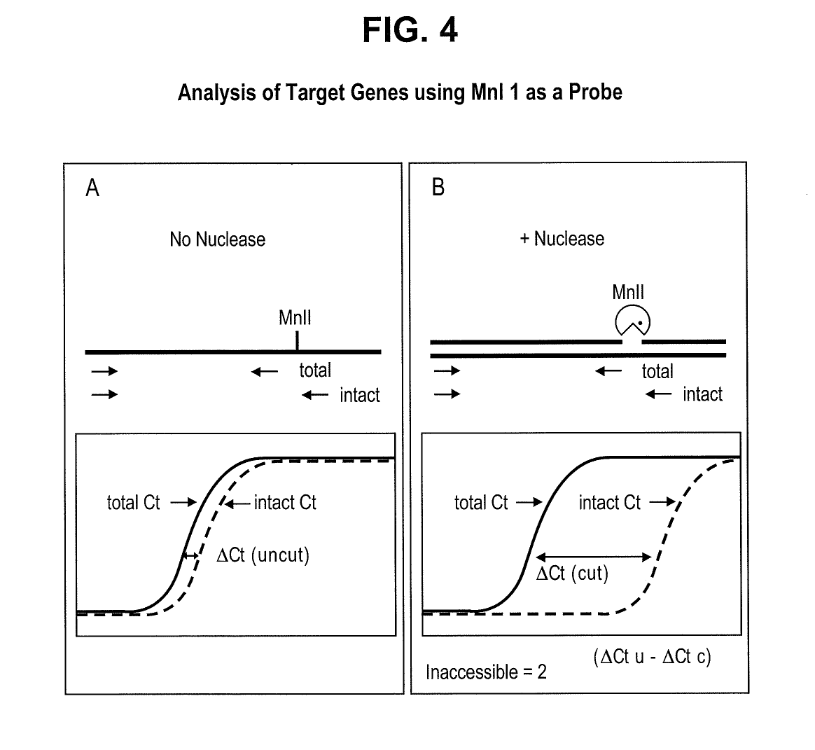

FIG. 4 illustrates a schematic for analysis of DNA isolated from cells when MnlI is used as a chromatin structure probe. The assay in this case is performed in parallel with a “no nuclease” control. Two primer sets are used for each gene. One primer set does not span any MnlI digestion sites, whereas the other primer set spans at least one MnlI site. Cells treated with the permeabilization reagent, but no nuclease, result in full amplification (i.e. all copies amplified) from both primer sets and thus should have a similar Ct value, as illustrated in FIG. 4, left graph. The delta Ct value (Total Ct minus intact Ct) reflects the difference in Ct values between these primer sets in uncut DNA based on primer peculiarities.

In contrast, in cells that have been treated with MnlI, accessible genes should be cleaved at the MnlI site. The primer set that does not span a MnlI site will still amplify all the DNA molecules (acting as a measure for the total number of copies) and the Ct will thus reflect the quantity of total copies of DNA. The other primer set (spanning at least one MnlI site) will only be able to amplify uncut or intact sequences. Depending on the extent of digestion, the Ct value will shift and increase in proportion to the amount of digestion. Delta Ct can then be calculated by subtracting intact Ct from total Ct, and if the DNA is accessible this will result in a negative number. The percentage of MnlI sites that are inaccessible is calculated by the equation shown in FIG. 4.

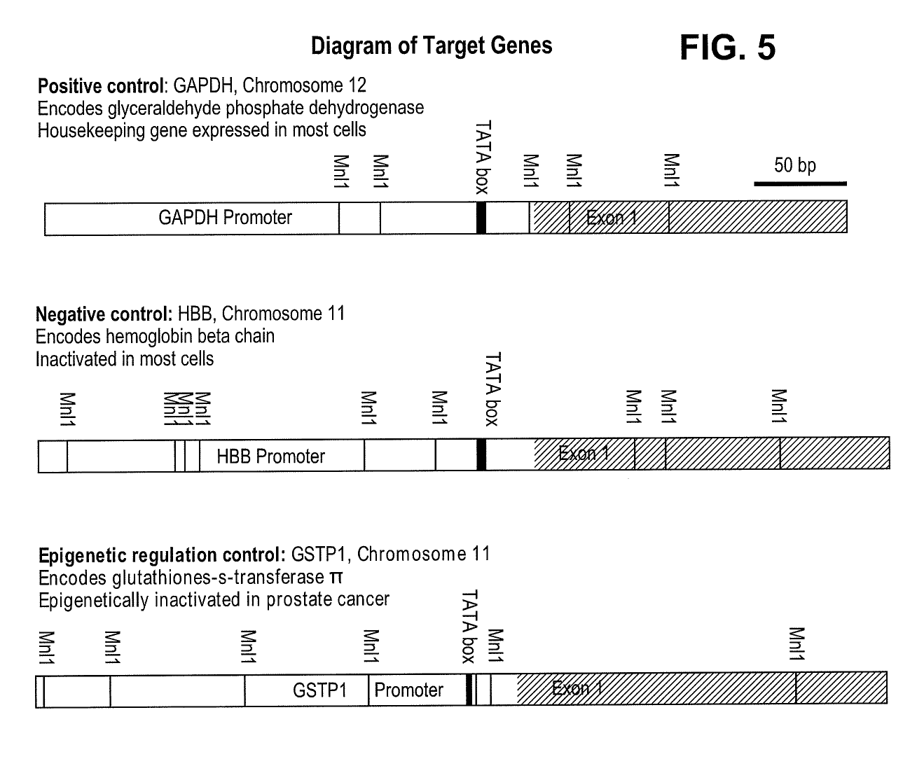

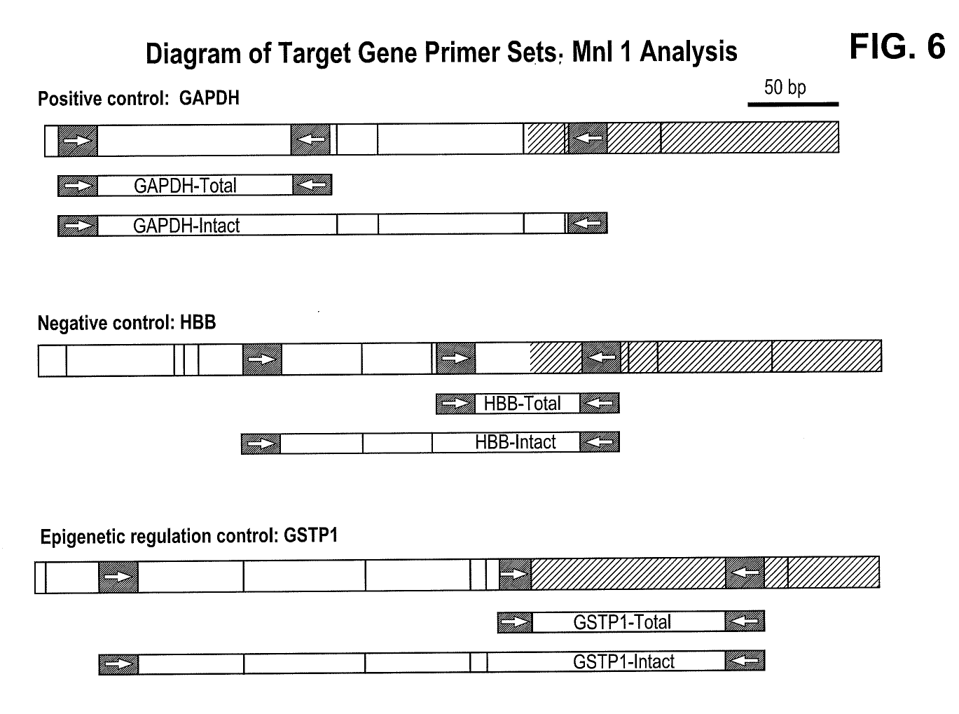

Application of this technique to different types of DNA regions is illustrated in FIGS. 5-6. FIG. 5 shows representations of at least portions of the GAPDH, HBB, and GSTP1 genes. Mnl I, the TATA box, promoter regions, and exons are shown. GAPDH was used as a positive control. GAPDH is a housekeeping gene that is expressed in most cell lines and should be accessible. The hemoglobin beta chain gene was chosen as a negative control. Hemoglobin is not expressed in most cells, is epigenetically silenced, and is expected to be in an inaccessible chromatin structure. Finally Glutathione-s-transferase pi, or GSTP1, which is epigenetically inactivated in prostate cancer, was analyzed. FIG. 6 shows the regions that can be targeted for amplification. For each gene, a total primer set that does not span any Mnl I sites and that will amplify all DNA strands is used. An “intact” primer set that spans at least one (e.g., two to four) MnlI sites and that will only amplify DNA strands that are not cut at these sites is also used.

### Example 2

### Data Using WI in Hela Cells

This example shows the results of data generated using an experiment approach as outlined above.

### Materials and Methods

Chemicals. L-α-Lysophosphatidylcholine (lysolecithin) was purchased from Sigma-Aldrich. MnlI, DNase I, BSA and proteinase K were purchased from New England Biolabs. RNase A was purchased from Qiagen. Tissue culture plates were purchased from VWR. iQ SYBR was from Bio-Rad.

Treatment of cells. Cells grown in 24-well plates were treated when they reached 90% confluence. The culture media was aspirated and 100 ul of a permeabilization/digestion buffer was gently layered on the cells. For cells treated with MnlI the permeabilization/digestion buffer consisted of lysolecithin, NaCl, Tris-HCl, MgCl₂, DTT, BSA and MnlI. For cells treated with DNase I the permeabilization/digestion buffer consisted of lysolecithin, Tris-HCl, MgCl₂, CaCl2 and DNase I. The permeabilized cells were then incubated at 37° C. for 1 hour. Following incubation 25 ul of lysis/stop solution (100 mM Tris-HCl pH 7.4, 100 mM NaCl, 100 mM EDTA, 5% N-lauroylsarcosine (w/v), 80 ug/ml RNase A and 3 mg/ml proteinase K) was added to the permeabilization/digestion buffer and the cell lysates were incubated at 37° C. 10 minutes.

Isolation of DNA. The cell lysates were collected and the DNA was isolated using a commercial nucleic acid purification kit (Aurum, Bio-Rad) following standard procedures. The DNA was eluted in 100 ul and the concentration of DNA was determined using a NanoDrop 1000 spectrophotometer (Thermo Scientific). The samples were then diluted to a concentration of 1-10 ng DNA per ul.

Genes analyzed. We analyzed the promoter region of three human genes: glycealdehyde-3-phosphate dehydrogenase (GAPDH), hemoglobin, beta (HBB) and glutathione-s-transferase π (GSTP1). GAPDH is a housekeeping gene; it is expressed in most cells and is expected to have an accessible chromatin structure. HBB is not expressed in most cells and is expected to have an inaccessible chromatin structure. GSTP1 is epigenetically inactivated in prostate cancer. In the non-cancerous prostate cell line, RWPE-1, GSTP1 is in an accessible chromatin structure and is expressed, in the cancerous prostate cell line LNCaP GSTP1 is in an inaccessible chromatin structure and is not expressed (Okino, S. T., et al., Chromatin changes on the GSTP1 promoter associated with its inactivation in prostate cancer. Mol Carcinog, 2007. 46(10): p. 839-46). GSTP1 mRNA is also highly expressed in the Hela and HCT15 cell lines; in the PC3 cell line GSTP1 mRNA is expressed at a level approximately half that found in Hela and HCT15 cells.

Real-time PCR. DNA samples isolated from cells treated with MnlI were analyzed using each primer set in a PTC-200 thermal cycler fitted with a Chromo4 continuous fluorescence detector (MJ Research). Each reaction was 20 ul in volume and contained 50 ng of DNA, 500 nM of each primer and 10 ul of iQ SYBR green supermix (Bio-Rad). PCR conditions were 96° C. for 10 minutes; 40 cycles of 96° C. for 30 seconds and 68° C. for 1 minute; 5 minutes at 72° C.; melt curve from 72° C. to 98° C. at 0.2° C. intervals holding for 5 seconds at each step. DNA samples isolated from cells treated with DNase I were analyzed in a CFX96 Real-Time PCR Detection System (Bio-Rad). Each reaction was 20 ul in volume and contained 5 ng of DNA, 500 nM of each primer and 10 ul of iTaq Fast SYBR green supermix with ROX (Bio-Rad). PCR conditions were 95° C. for 5 minutes; 40 cycles of 95° C. for 30 seconds and 72° C. for 1 minute; 5 minutes at 72° C.; melt curve from 72° C. to 95° C. at 0.2° C. intervals holding for 5 seconds at each step.

Data analysis. To determine the level of inaccessible chromatin using MnlI as a nuclease probe we performed the following calculations. ΔCt (uncut) was calculated as (intact Ct−total Ct) when analyzing DNA isolated from cells that were treated with digestion/permeabilization buffer that lacked MnlI. ΔCt (cut) was calculated as (intact Ct−total Ct) when analyzing DNA isolated from cells that were treated with MnlI. The level of inaccessible chromatin was calculated as 2̂(ΔCt (uncut)−ΔCt (cut)) (see FIG. 4).

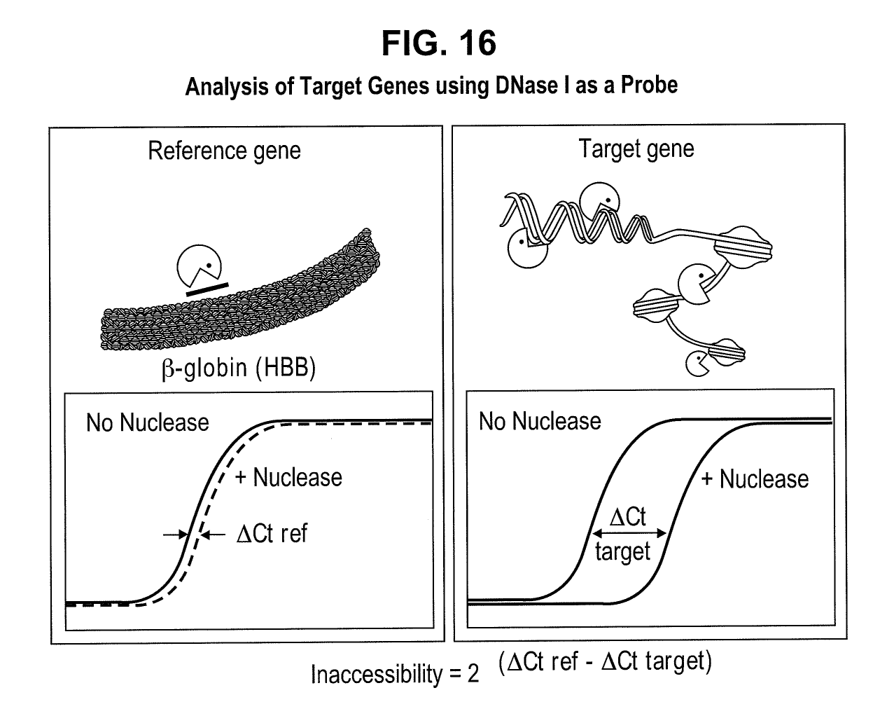

To determine the level of inaccessible chromatin for GAPDH and GSTP1 using DNase I we could not use the same calculations that we used when analyzing MnlI because DNase I cuts within both the total and intact amplification regions in accessible chromatin. We found that the HBB promoter is highly resistant to DNase I digestion, we used HBB ΔCt as an internal reference standard that reflects the low level of digestion of inaccessible chromatin (illustrated in FIG. 16). ΔCt (ref) was calculated as (HBB+nuclease Ct−HBB no nuclease Ct). ΔCt (target) was calculated as GAPDH/GSTP1+nuclease Ct−GAPDH/GSTP1 no nuclease Ct. The level of inaccessible chromatin of the GAPDH or GSTP1 target genes was calculated as 2̂(ΔCt (ref)−ΔCt (target)).

### Results

Hela cells were incubated in either: digestion buffer but no nuclease, digestion buffer that lacked lysolecithin (the cell permeabilization reagent) and nuclease, or a complete digestion buffer with lysolecithin and nuclease (MnlI [M] or DNase I [D]).

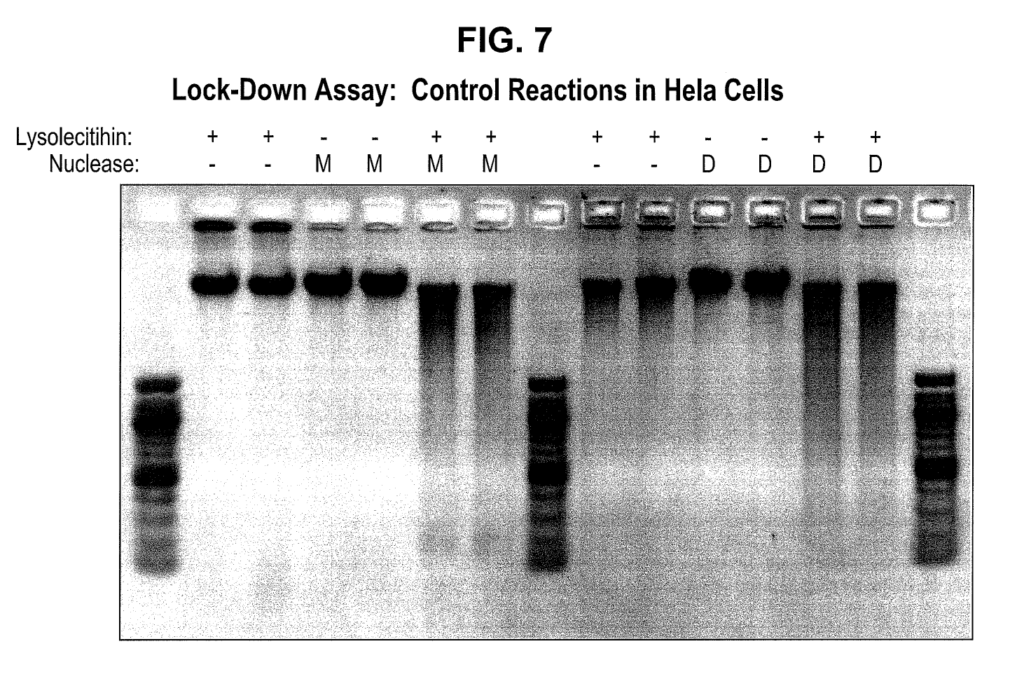

Genomic DNA (gDNA) was then isolated from the cells and submitted to agarose gel electrophoresis (see FIG. 7). The data indicated that lysolecithin and the nuclease were required to digest gDNA. Only slight gDNA digestion was detected in reactions lacking either of lysolecithin or the nuclease. This indicates that in cells permeabilized by lysolecithin, digestion of chromatin by endogenous nucleases is not significant and that nuclease present in the digestion buffer does not enter the cell in the absence of lysolecithin.

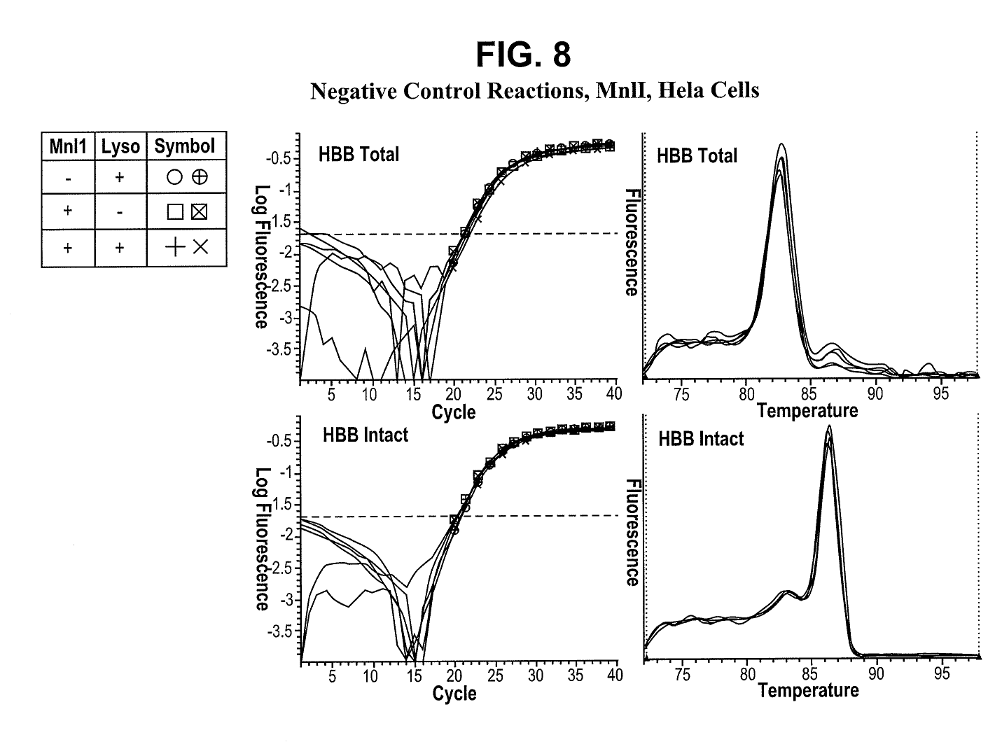

Subsequently, the DNA samples isolated from MnlI treated cells were analyzed by real-time PCR using the primer sets as shown in FIG. 6. As shown in FIG. 8, the negative control reactions analyzed the hemoglobin beta chain gene (which is silenced in Hela cells). For both the total and intact primer sets, the Ct values were about the same. This indicates that this gene region was in an inaccessible chromatin structure in Hela cells.

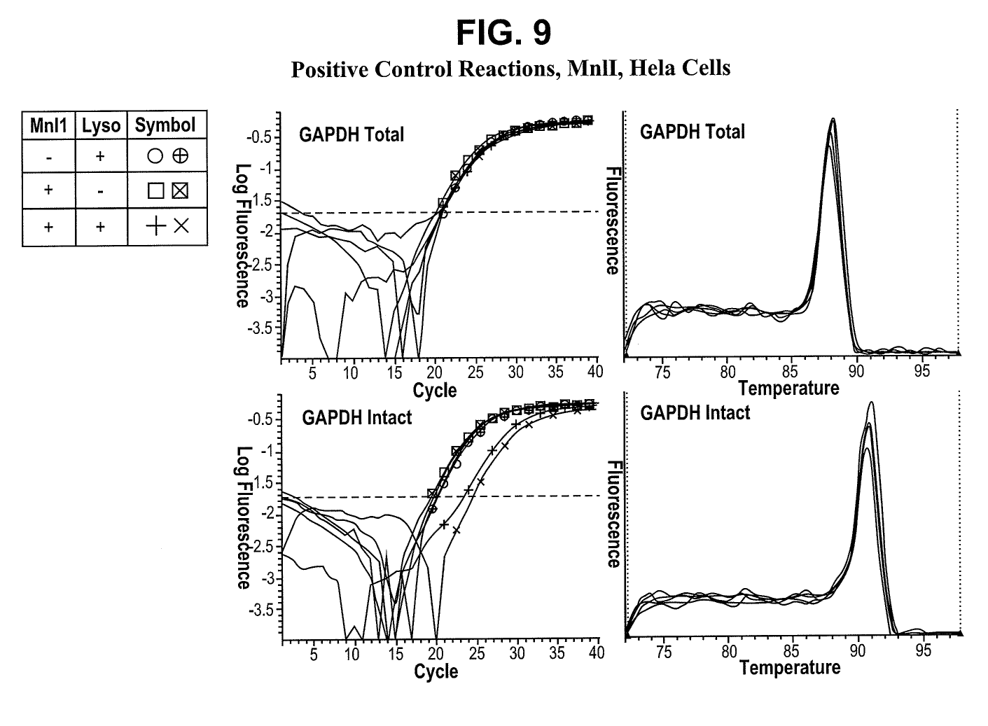

FIG. 9 shows the results for GAPDH, the positive control. The Ct values for GAPDH were about the same as obtained for HBB in the negative control, but amplification using the “intact” primer set was shifted to the right, indicating less intact than “total” DNA. This means that MnlI accessed and digested at its recognition sites in the GAPDH promoter. We infer, therefore, that the GAPDH promoter was in accessible chromatin in Hela cells.

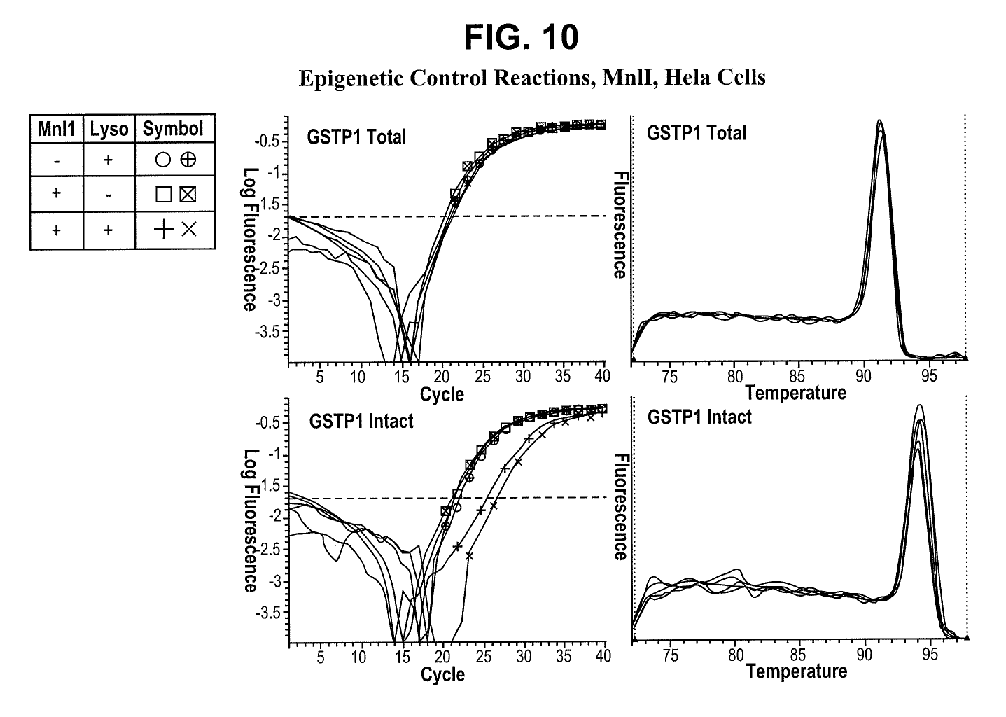

FIG. 10 shows results from analysis of GST pi. GST pi is inactivated in prostate cancer and is expressed in Hela cells. The Ct value of the complete reactions are right shifted in the intact primer set reactions, but not in the total primer set reactions. This indicates that GST pi is in an accessible chromatin structure in Hela cells.

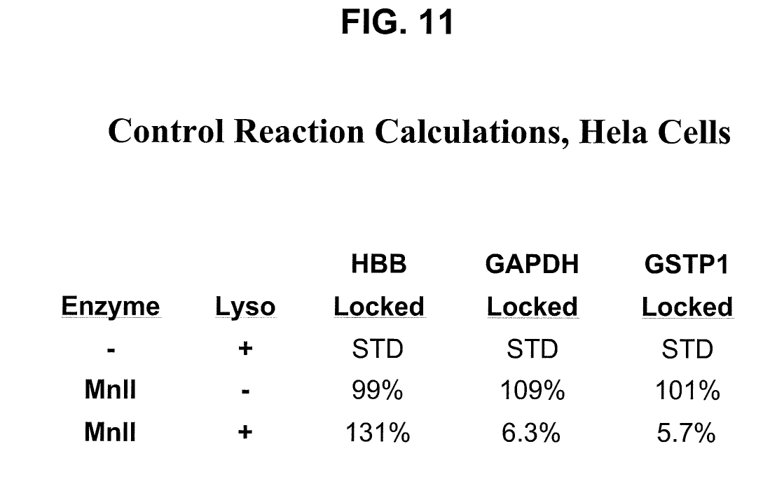

FIG. 11 summarizes the above-described data in a table. The calculated values are the percentage of copies that were intact (and therefore inaccessible to the nuclease) compared to the total number of copies as determined by the “total” primer set.

### Example 3

### Data Using MnlI in Prostate Cancer Cells

The methods described above were also applied to different cell types to determine whether accessibility was related to expression for GSTP1. Two cell types were used: RWPE-1 cells, which are derived from non-cancerous human prostate, and in which GSTP1 is highly expressed; and LNCaP cells, which are derived from cancerous human prostate, and in which GSTP1 is silenced by epigenetic modifications.

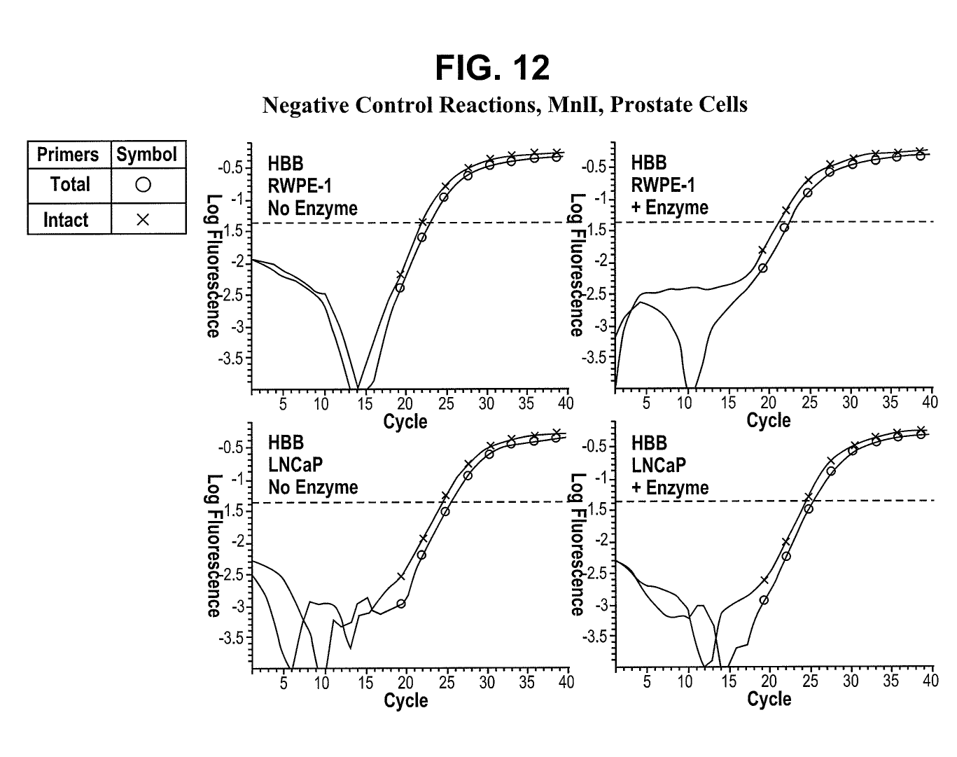

The methods of analysis of these cells were essentially as described above. FIG. 12 shows the control reactions (i.e., for the hemoglobin gene). In both cell lines, the delta Ct in the samples with no enzyme are about the same as those with enzyme. Therefore the delta Cts were about 0, indicating that this gene was inaccessible in both cell lines.

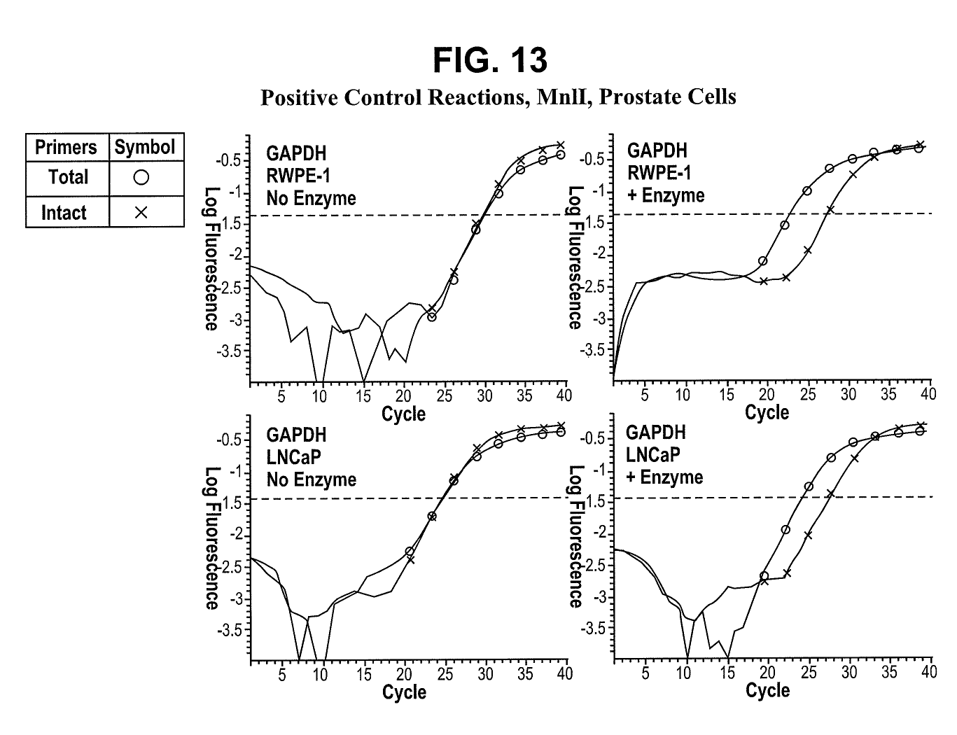

FIG. 13 shows the results of the positive control reactions analyzing the GAPDH gene. For reactions without enzyme, both the total and intact Cts were about the same. However, for reactions with enzyme, the intact Ct shifted to the right, indicating that there was less intact DNA and therefore that the GAPDH gene was in an accessible chromatin structure.

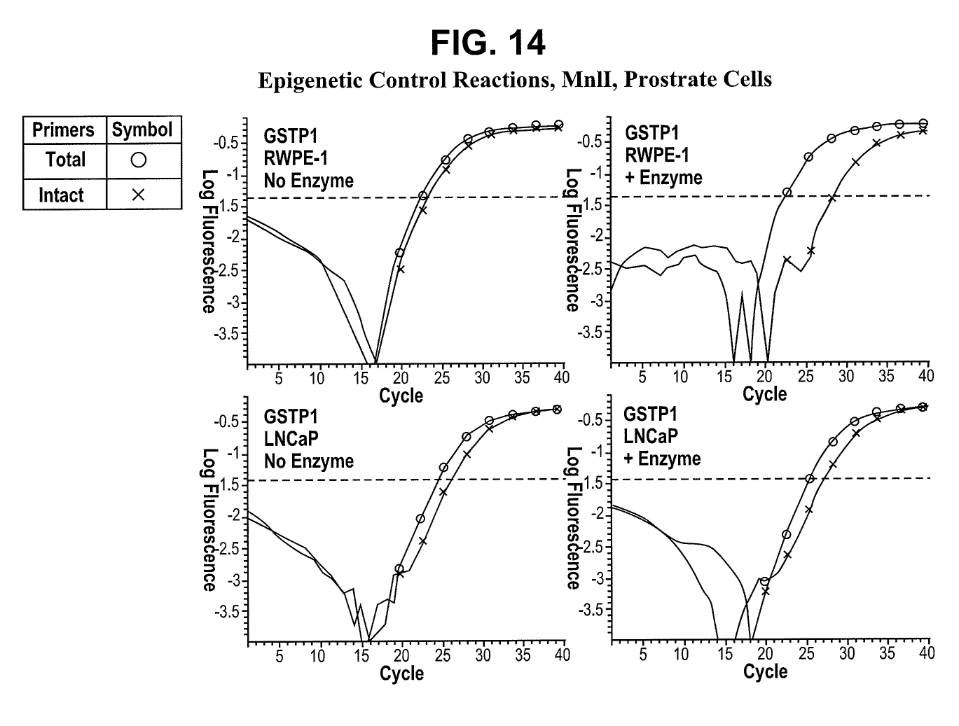

FIG. 14 shows data from the analysis of the GST pi gene. In the non-cancerous RWPE-1 cell line, the intact Ct shifted to the right in the presence of enzyme. In contrast, in the cancerous LNCaP cell line the intact Ct did not change to an appreciable extent. This indicates that GSTP1 was in accessible chromatin in RWPE-1 cells and in inaccessible chromatin in LNCaP cells.

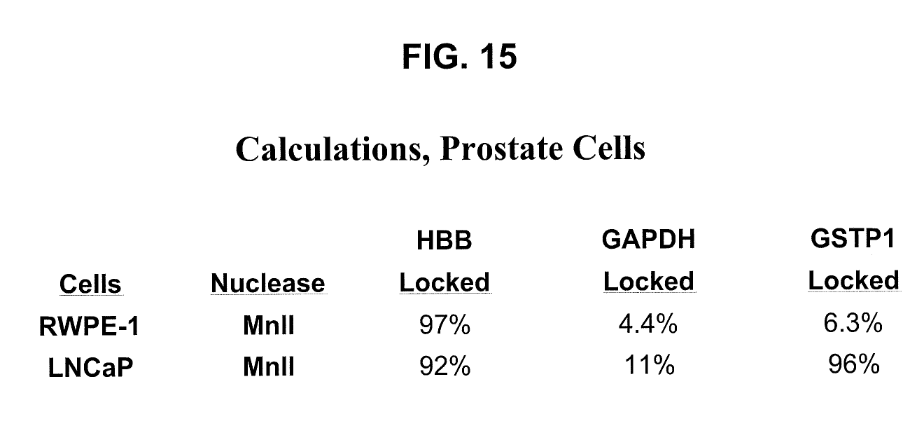

These experiments were repeated four times. A summary of the results are shown in FIG. 15, again with the percentage of copies that were inaccessible (or “locked”) calculated.

### Example 4

### Data Using DNase I

This example shows data from use of the method where DNase I, rather than MnlI, was the nuclease. In this example we analyzed four human cancer cell lines: Hela (cervical cancer), PC3 (prostate cancer), LNCaP (prostate cancer) and HCT15 (colon cancer). We also analyzed the same genes that we analyzed in the MnlI example: HBB, GAPDH and GSTP1.

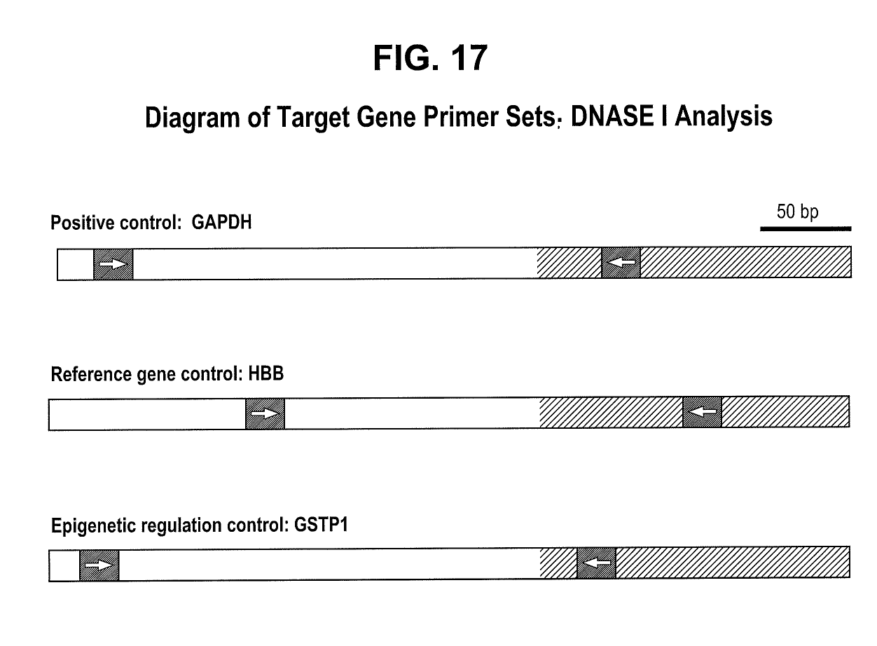

Because DNase I does not digest DNA at defined sites we could not use the same data analysis strategy we used as when MnlI was the chromatin structure probe. Instead we utilized an epigenetically silenced reference gene (HBB) as an internal control that reflects the extent of digestion of inaccessible chromatin. Using this strategy, the chromatin structure of specific target genes can be determined as detailed in FIG. 16. FIG. 17 is a schematic depiction of the genes analyzed and the location of the primers used to assess chromatin structure.

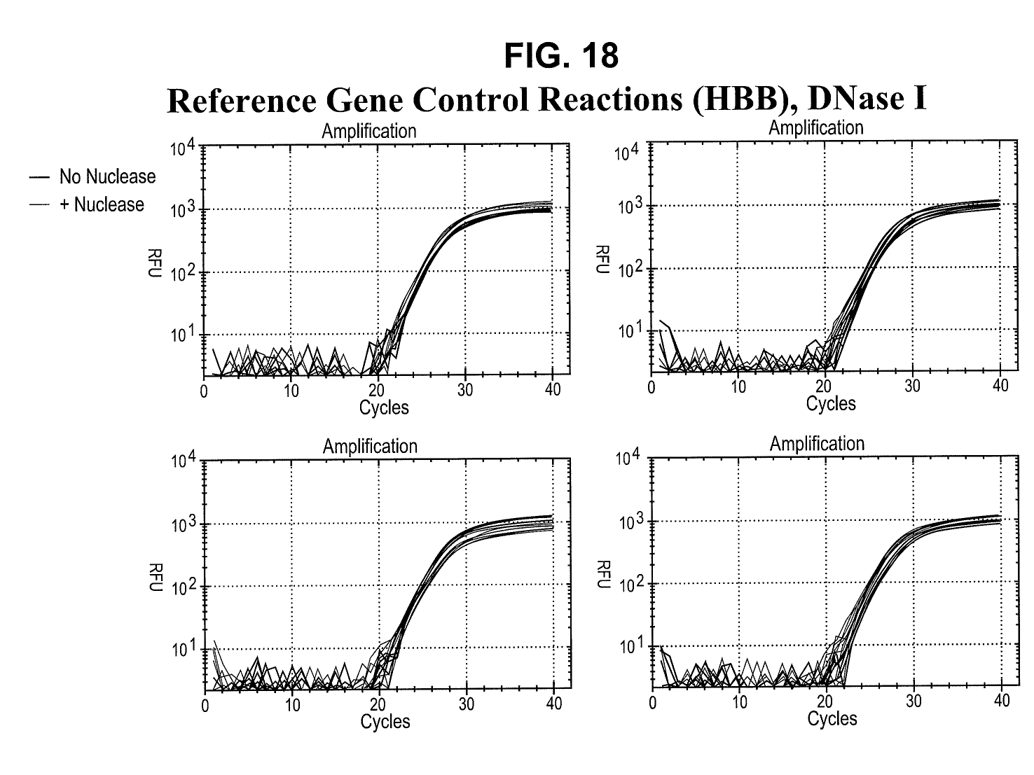

As shown in FIG. 18, DNase I did not digest HBB to an appreciable extent in all cell lines (the no nuclease and + nuclease curves almost overlap). This result indicates that HBB is in an inaccessible chromatin configuration in all cell lines and validates its use as a reference gene control. These results are also expected because HBB mRNA is not expressed in any of these cell lines (data not shown).

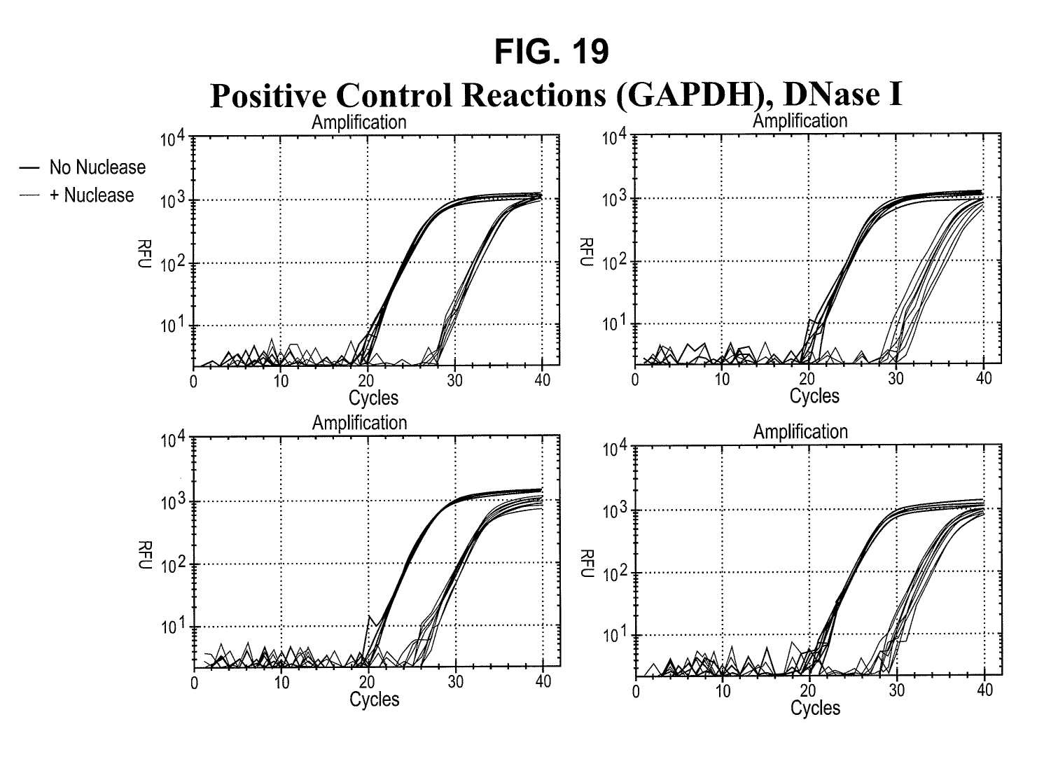

Our analysis of the GAPDH gene is shown in FIG. 19. In all cell lines we observe that the + nuclease curve is significantly right-shift relative to the no nuclease curve. This implies that GAPDH is in an accessible chromatin structure in all cell lines. These results are expected because GAPDH is a “housekeeping” gene that is expressed in most, if not all, cell lines (data not shown).

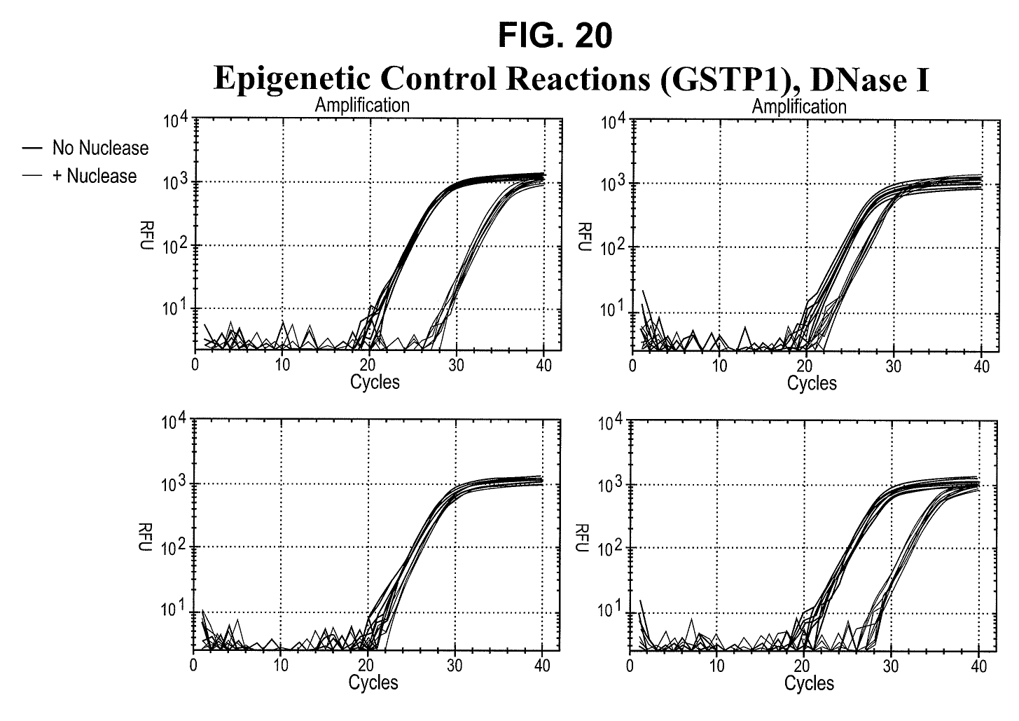

GSTP1 mRNA is expressed at different levels in the analyzed cell lines. Hela and HCT15 cells express high amounts of GSTP1 mRNA, PC3 cells express a moderate amount (about half of that found in Hela and HCT15 cells) and LNCaP cells do not express GSTP1 mRNA to an appreciable extent (data not shown). Our analysis of GSTP1 chromatin structure is shown in FIG. 20. In Hela and HCT15 cells we observe that the + nuclease curve is significantly right-shift relative to the no nuclease curve implying that GSTP1 is in an accessible chromatin structure. In PC3 cells we observe that the + nuclease curve is moderately right-shift relative to the no nuclease curve implying that GSTP1 is in an a partially accessible chromatin structure. Finally, in LNCaP cells we observe that the + nuclease curve and the no nuclease curve almost overlap. This implies that GSTP1 is in an inaccessible chromatin configuration in LNCaP cells. Significantly, we observe an excellent correlation between GSTP1 chromatin structure and GSTP1 mRNA expression in all cell lines analyzed.

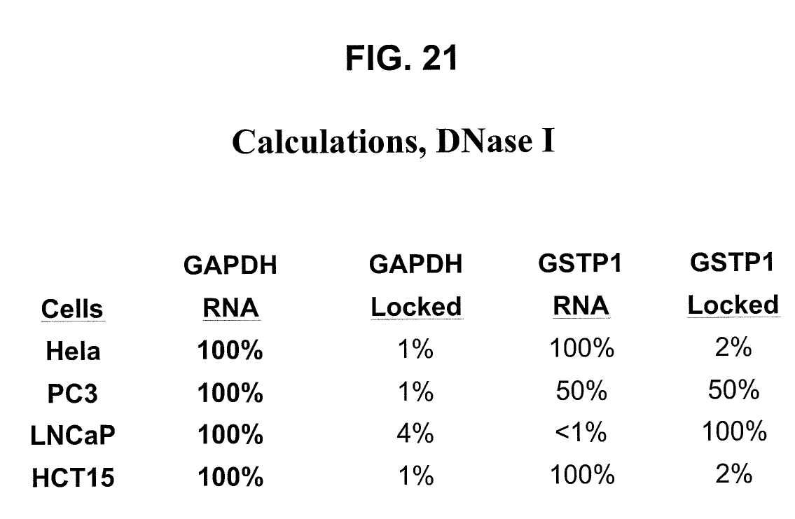

FIG. 21 shows a summary of results. The percentage of inaccessible DNA was calculated as described in FIG. 16 and is shown as “locked” chromatin. The relative level of GAPDH and GSTP1 RNA expression was calculated by analyzing mRNA isolated from the different cell lines by qRT-PCR by standard procedures. The results indicate that target gene chromatin structure correlates well with the level of target gene expression. This implies that the assay described represents a useful tool to assess epigenetic effects on gene expression.

### Summary

We have developed a rapid and sensitive assay that can assess chromatin structure in cultured cells. Our assay offers several advantages over other chromatin structure assays (Okino, S. T., et al., Mol Carcinog 46(10):839-46 (2007); Okino, S. T. and J. P. Whitlock, Jr., Mol Cell Biol 15(7): p. 3714-21 (1995); Rao, S., E. Procko, and M. F. Shannon, J Immunol 167(8):4494-503 (2001); Kilgore, J. A., et al., Methods 41(3):320-32 (2007)). Most significantly, in our procedure we digest chromatin in permeabilized cells in one step in situ. Other procedures require nuclei isolation which requires more labor, more time and more cells as starting material. In addition, our primer set design results in a more accurate and internally controlled assessment of chromatin accessibility.

### Example 5

### Variation to Assess DNA Methylation

Another application of the present invention is to determine the DNA methylation status of inaccessible chromatin regions. DNA methylation refers to the natural methylation of cytosine at the 5-position to generate 5-methyl cytosine. DNA methylation is implicated in regulating gene activity and chromatin structure; aberrant patterns of DNA methylation are associated with human pathologies and specific disease states. In this variation the previously described procedure is followed up to, but not including, the step of PCR amplification. The purified genomic DNA is then modified by bisulfite, using standard procedures, such that cytosines that are unmethylated are converted to uracil and 5-methyl cytosines are unchanged. A primer set that targets a specific DNA region is then used to amplify bisulfite-modified DNA purified from cells that have been treated with a nuclease. Thus, only DNA that was originally in an inaccessible chromatin configuration, and thus not cleaved by the DNA modifying agent, can be amplified. The amplified DNA is then analyzed for DNA methylation by standard procedures such as melt curve analysis, high resolution melt analysis, bisulfite-DNA sequencing, digital PCR, methylation-specific PCR, pyrosequencing etc. Such analysis can determine the DNA methylation status of inaccessible chromatin regions and can provide valuable information regarding specific patterns of DNA methylation associated with epigenetic gene inactivation.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for analyzing chromosomal DNA, the method comprising,
a. simultaneously:
i. permeabilizing or disrupting a cell membrane of a cell; and
ii. contacting the cell with a DNA cleaving or modifying agent under conditions such that the agent cleaves or modifies the genomic DNA in the cell, wherein different regions of the genomic DNA are cleaved or modified to a different extent by the agent, thereby generating cleaved and intact DNA regions, or modified and unmodified DNA regions; and
b. detecting a quantity of at least one intact or unmodified or modified DNA region or cloning, isolating, or nucleotide sequencing at least one intact or unmodified or modified DNA region.

2. The method of claim 1, comprising correlating the quantity of at least one intact or unmodified or modified DNA region to chromatin structure of the DNA region in the cell.

3. The method of claim 1, comprising
detecting a quantity of at least a first chromosomal intact or unmodified or modified DNA region and the second chromosomal intact or unmodified or modified DNA region; and
comparing the quantity of the first and second DNA regions.

4. The method of claim 1, comprising quantifying the number of intact copies of a first chromosomal DNA region and a second chromosomal DNA region, thereby assessing the relative accessibility of the first and second DNA regions to the DNA cleaving agent.

5. The method of claim 1, wherein the genomic DNA is isolated after the contacting step and before the detecting step.

6. The method of claim 1, wherein the cells are permeabilized and contacted with the DNA cleaving or modifying agent while the cells are directly or indirectly adherent to an artificial culture surface.

7. The method of claim 1, wherein the permeabilizing step comprises contacting the cell with an agent that permeabilizes the cell membrane.

8. The method of claim 7, wherein the agent that permeabilizes the cell membrane is a lysolipid.

9. The method of claim 1, wherein the cell is contacted with a DNA cleaving agent, and the DNA cleaving agent is an enzyme.

10. The method of claim 9, wherein
the DNA cleaving agent is a DNA cleaving enzyme;
the modifications in the genomic DNA are sites where the DNA has been cleaved; and
the detecting step comprises quantifying the amount of intact copies of a DNA region after cleavage.

11. The method of claim 10, wherein the detecting step comprises quantitative amplification.

12. The method of claim 9, wherein the DNA cleaving agent is selected from a DNase and a restriction enzyme.

13. The method of claim 1, wherein
the detecting step comprises quantifying at least a portion of a target DNA region and at least a portion of a control DNA region, wherein the control DNA region comprises a sequence that is either
i. accessible in essentially all cells of an animal; or
ii. inaccessible in most cells of an animal.

14. The method of claim 1, wherein the control DNA region is quantitatively amplified using each of:
primers that prime amplification of a portion of the control DNA region that does not include a potential modification site of the DNA modifying agent; and
primers that prime amplification of a portion of the control DNA region that does include at least one potential modification site of the DNA modifying agent.

15. The method of claim 1, wherein the DNA methylation of the DNA region is determined.

16. The method of claim 15, wherein step a. comprises contacting the DNA with a DNA cleaving agent, and step b. comprises contacting the intact DNA with bisulfite.

17. The method of claim 16, further comprising determining the melting temperature of the bisulfite-treated DNA and correlating the melting temperature to the presence, absence, or extent of DNA methylation.

18. The method of claim 1, wherein step b comprises preparing a library of:
intact DNA regions; or
modified DNA regions; or
unmodified DNA regions.

19. The method of claim 1, wherein step b comprises contacting at least one intact or unmodified or modified DNA region to a library of polynucleotides under conditions to allow for hybridization of the DNA region to one or more members of the library and detecting hybridization of the DNA region to the one or more members.

20. The method of claim 1, wherein step b comprises nucleotide sequencing at least one intact or unmodified or modified DNA region.

21. A kit for determining the accessibility of a locus on a chromosome to a DNA modifying agent, the kit comprising,
a cell membrane permeabilizing or disrupting agent;
a restriction enzyme or a DNase.
